(19) United States
(12) Patent Application Publication
(10) Pub. No.: US 2020/0338056 A1
(43) Pub. Date: Oct. 29, 2020

(54) INDUCTION OF SENESCENCE USING PROTON PUMP INHIBITORS

(71) Applicant: THE METHODIST HOSPITAL SYSTEM, Houston, TX (US)

(72) Inventors: John P. COOKE, Houston, TX (US); Gautham YEPURI, Houston, TX (US); Yohannes Tsegai GHEBRE, Houston, TX (US)

(73) Assignee: THE METHODIST HOSPITAL SYSTEM, Houston, TX (US)

(21) Appl. No.: 16/086,304

(22) PCT Filed: Mar. 23, 2016
(86) PCT No.: PCT/US2017/023838

§ 371 Date: Sep. 18, 2018

Related U.S. Application Data
(60) Provisional application No. 62/312,333, filed on Mar. 23, 2016.

(51) Int. Cl. A61K 31/4439; C12N 5/071; C12N 5/077; G01N 33/68

(52) U.S. Cl. CPC A61K 31/4439; C12N 5/0629; C12N 5/0656; A61K 9/0014; G01N 33/6872; C12N 2501/999; G01N 33/6893

(57) ABSTRACT

Provided herein are proton pump inhibitors that promote cellular senescence, methods of using the proton pump inhibitors to promote cellular senescence and compositions and kits comprising the proton pump inhibitors. Also provided are methods of screening for candidate agents that promote senescence or inhibit senescence.

### CROSS-REFERENCE TO RELATED APPLICATIONS

This application claims priority to U.S. Provisional Application No. 62/312,333, filed Mar. 23, 2016, which is incorporated by reference herein in its entirety.

### STATEMENT REGARDING FEDERALLY SPONSORED RESEARCH

This invention was made with government support under grant numbers 1U01HL100397 and K01HL118683 awarded by the National Institute of Health. The government has certain rights in the invention.

### BACKGROUND

Proton pump inhibitors (PPIs), such as esomeprazole (NEXIUM®, Astrazeneca Ab Corporation Sweden), are widely used drugs for the treatment of gastroesophageal reflux disease. Other PPIs include rabeprazole, omeprazole, lansoprazole, dexlansoprazole, pantoprazole, and ilaprazole. In the United States, these drugs may be prescribed, but many are now sold over the counter, and thus medical supervision is not required. Although these agents are effective, they were never approved by regulatory authorities for long-term use. Furthermore, evidence suggests that up to about 70% of PPI use may be inappropriate. Recent large and well-controlled epidemiological and retrospective studies have found associations between the use of PPIs and an increased prevalence of myocardial infarction, renal failure, and dementia. However, in the absence of a mechanism and without evidence of causality, global regulatory authorities have not restricted the use of PPIs.

### BRIEF SUMMARY

Provided herein are proton pump inhibitors that promote cellular senescence, methods of using the proton pump inhibitors to promote cellular senescence and compositions and kits comprising the proton pump inhibitors. Also provided are methods of screening for candidate agents that promote senescence or inhibit senescence.

### BRIEF DESCRIPTION OF THE DRAWINGS

FIGS. 1A, 1B, 1C, 1D, 1E, and 1F show esomeprazole impairs proteostasis. FIG. 1A is a graph showing the intensity of pHrodo Green AM fluorescence, which is inversely proportional to lysosomal pH (n=4). FIG. 1B is a graph showing acid phosphatase assay (n=4). FIG. 1C shows fluorescent images and 1E is a graph showing intracellular cathepsin-B activity assessed by Magic Red fluorescence dye (n=4). FIG. 1D shows fluorescent images and 1F is a graph showing intracellular protein aggregates assessed by PROTEOSTAT assay (fluorescent staining in upper panel and corresponding phase-contrast image on lower panel) and quantification (n=4). *P<0.05 vs vehicle (DMSO). ESO indicates esomeprazole.

FIGS. 2A, 2B, 2C, 2D, 2E, 2F, 2G, 2H, 2I, 2J, 2K, and 2L show esomeprazole impairs endothelial function. FIG. 2A shows images and 2C is a graph showing superoxide anion generation assessed by dihydroethidium staining (n=4). FIG. 2B shows images and 2D is a graph showing nitric oxide generation assessed by diamino fluorescein 2-diacetate (DAF-2DA) staining (n=4). FIG. 2E is a graph showing total nitrate/nitrite levels assessed by Greiss reaction (n=6). FIG. 2F is a graph showing measurement of cell proliferation using real-time cell analyzer, which generates cell index values represented as area under curve (AUC; n=5). FIG. 2G is a graph showing cell proliferation assessed by 5-bromo-2′-deoxyuridine (BrdU) assay (n=8). FIG. 2H is a graph showing p21 mRNA expression using reverse transcription polymerase chain reaction (n=4). FIG. 2I shows images and FIGS. 2J, 2K, and 2L are graphs showing angiogenic capacity of endothelial cells reflected by network formation in growth factor depleted matrigel. *P<0.05 vs vehicle (DMSO). DHE, dihydroethidium; ESO, esomeprazole; GAPDH, glyceraldehyde 3-phosphate dehydrogenase; and NO, nitric oxide.

FIGS. 3A, 3B, 3C, 3D, 3E, 3F, 3G, 3H, and 3I show proton pump inhibitors (PPIs) accelerate endothelial senescence. FIGS. 3A and 3D show images indicating senescent cell number detected by staining for senescence-associated β-galactosidase (SA-β-gal; top) and for SYTO-13 to detect cell nuclei for total cell count (bottom). FIGS. 3B, 3C, 3E and 3F, are graphs showing respective quantification for % positive SA-β-gal cells and average cell count per field (n=6). FIG. 3G is a gel image and 3H is a graph showing PAI-1 protein expression by Western blot analysis (n=3). FIG. 3I is a graph showing plasminogen activator inhibitor (PAI-1) mRNA expression quantified by reverse transcription polymerase chain reaction (n=6). *P<0.05 vs vehicle (DMSO). ESO indicates esomeprazole; and GAPDH, glyceraldehyde 3-phosphate dehydrogenase.

FIGS. 4A, 4B, 4C, 4D, 4E, 4F, and 4G, are graphs showing proton pump inhibitors reduce telomere length and expression of shelterin complex subunits. FIG. 4A is a graph showing relative telomere length assessed by monochrome multiplex quantitative polymerase chain reaction (PCR) in human microvascular endothelial cells (n=6). FIGS. 4B-4G are graphs showing expression of shelterin complex genes assessed by reverse transcription PCR (n=6). *P<0.05 vs vehicle (DMSO). ESO indicates esomeprazole; and GAPDH, glyceraldehyde 3-phosphate dehydrogenase.

FIG. 5 shows images at high power view of lysosomal distribution of pHrodo™ red in vehicle treated and esomeprazole (ESO) treated cells. Fluorescence is reduced in ESO treated cells consistent with an increase in lysosomal pH.

FIG. 6 is a graph showing esomeprazole does not impair NAG activity. β-N-Acetylglucosaminidase activity assay (n=4).

FIGS. 7A, 7B, 7C, and 7D are graphs showing esomeprazole decreases expression of genes related to NO signaling. Expression of DDAH1/2, eNOS and iNOS as detected by RT-PCR (n=4-6). *p<0.05 ESO vs vehicle (DMSO).

FIG. 8 is a graph showing esomeprazole reduces cell proliferation. Measurement of cell proliferation using real time cell analyzer which generates cell index (CI) values represented as area under curve. EC treated continuously with esomeprazole (ESO; 1 uM) for 3 passages (P4-6) manifested a reduction in cell proliferation by comparison to vehicle treated cells.

FIG. 9A shows images and 9B and 9C are graphs showing ranitidine does not accelerate endothelial senescence. FIG. 9A shows images indicating senescent cell number as detected by staining for senescence associated-β-galactosidase (SA-β-gal; upper panel) and for SYTO-13 to detect cell nuclei for total cell count (lower panel). FIGS. 9B and 9C are graphs showing respective quantification for % positive SA-β-gal cells and average cell count per field (n=4).

FIGS. 10A, 10B, 10C and 10D are graphs showing ESO increases expression of genes related to EndoMT signaling. EC expression of vwF, SMAD3, TWIST1 and COL1A1 by RT-PCR (n=4-6). *p<0.05 ESO vs vehicle (DMSO).

FIG. 11 shows images indicating esomeprazole accelerates EndoMT. Images of ECs treated with chronic exposure (3 passages; P4-P6) to ESO or vehicle, and then maintained for 81 days in endothelial growth medium without drugs or vehicle. Cells that were exposed to ESO during passage 4-6 manifest an acceleration of EndoMT in the absence of drug.

FIG. 12 is a gel image showing absence of telomerase activity in ECs. Telomerase activity as assessed by telomeric repeat amplification protocol assay (n=2).

### DETAILED DESCRIPTION

As described herein, chronic exposure to proton pump inhibition accelerates senescence in human endothelial cells (ECs) and other mammalian cells, which explains the association of adverse cardiovascular, renal, and neurological effects with the use of PPIs. Thus, provided herein is a method of screening for one or more agents that inhibit senescence. The method includes culturing mammalian cells with one or more proton pump inhibitors, wherein the proton pump inhibitors promote senescence of the mammalian cells; contacting the culture with one or more candidate agents; assaying the mammalian cells for one or more positive indicators and/or for one or more negative indicators of senescence. A decrease in the level of one or more positive indicators or an increase in the level of one or more negative indicators of senescence in the presence of the one or more candidate agent, as compared to a control culture lacking the one or more candidate agents, indicates the candidate agent inhibits senescence. Optionally, the mammalian cells are selected from the group consisting of endothelial cells, keratinocytes and fibroblast cells. Optionally, the mammalian cells are primary cells or immortalized cells. Optionally, the mammalian cells are selected from the group consisting of human umbilical venous endothelial cells, human aortic endothelial cells, human coronary artery endothelial cells, and human microvascular endothelial cells. Optionally, the one or more proton pump inhibitors is present in the culture at a concentration of 1 to 20 μmol/L. Optionally, the one or more proton pump inhibitors are selected from the group consisting of esomeprazole, lansoprazole, dexlansoprazole, omeprazole, pantoprazole, rabeprazole, and ilaprazole. Optionally, the culture is assayed by microscopy (e.g., fluorescence microscopy), fluorescence assay, colorimetric assay, sequencing, microarray, immunoassay, Western blot, Northern blot, qPCR, RT-PCR, or any combination thereof. Optionally, the positive indicator of senescence is selected from the group consisting of an increase in lysosomal pH, an increase in protein aggregation, an increase in superoxide anion, an increase in expression of cell cycle inhibitors, an increase in expression of plasminogen activator inhibitor, an increase in senescence-associated beta-galactosidase positive cells, an increase in elongated spindle-shaped cells, and any combination thereof. Optionally, the negative indicator of senescence is selected from the group consisting of a decrease in lysosomal enzyme activity, a decrease in nitric oxide levels, a decrease in nitrate levels, a decrease in activity of the NO synthase pathway, a decrease in replicative capacity of the cells, a decrease in angiogenic capacity, a change in morphology, a decrease in telomere length, reduced expression of the shelterin complex, a decrease in the mitotic index, and any combination thereof. Optionally, the negative indicator of senescence is a decrease in replicative capacity of the cells, a decrease in telomere length or a combination thereof. Optionally, the candidate agent is a peptide, nucleic acid, small molecule or any combination thereof. Optionally, the method includes administering the candidate agent that inhibits senescence to a subject. Optionally, the candidate agent treats an age-related disease or disorder in the subject.

Also provided is a method of screening for one or more agents that promote senescence. The method includes providing a first culture of mammalian cells and one or more proton pump inhibitors, wherein the proton pump inhibitors promote senescence of the mammalian cells; assaying the first culture for one or more positive and/or negative indicators of senescence; providing a second culture of mammalian cells and one or more candidate agents; assaying the second culture of mammalian cells for the same positive and/or negative indicators of senescence. Detection of one or more of the same positive and/or negative indicators of senescence in the second culture as compared to the first culture indicating the one or more candidate agents promotes senescence. Optionally, the mammalian cells are selected from the group consisting of endothelial cells, keratinocytes and fibroblast cells. Optionally, the mammalian cells are primary cells or immortalized cells. Optionally, the mammalian cells are selected from the group consisting of human umbilical venous endothelial cells, human aortic endothelial cells, human coronary artery endothelial cells, and human microvascular endothelial cells. Optionally, the one or more proton pump inhibitors in the first and second cultures is present in a concentration of 1 to 20 μmol/L. Optionally, the one or more proton pump inhibitors are selected from the group consisting of lansoprazole, dexlansoprazole, omeprazole, esomeprazole, pantoprazole, rabeprazole, and ilaprazole. Optionally, the first and second cultures are assayed by microscopy (e.g., fluorescence microscopy), fluorescence assay, colorimetric assay, sequencing, microarray, an immunoassay, Western blot, Northern blot, qPCR, RT-PCR, or any combination thereof. Optionally, the positive indicator of senescence is selected from the group consisting of an increase in lysosomal pH, an increase in protein aggregation, an increase in superoxide anion, an increase in expression of cell cycle inhibitors, an increase in expression of plasminogen activator inhibitor, an increase in senescence-associated beta-galactosidase positive cells, and any combination thereof. Optionally, the negative indicator of senescence is selected from the group consisting of a decrease in lysosomal enzyme activity, a decrease in nitric oxide levels, a decrease in nitrate levels, a decrease in activity of the NO synthase pathway, a decrease in replicative capacity of the cells, a decrease in angiogenic capacity, a change in morphology, a decrease in telomere length, reduced expression of the shelterin complex, a decrease in the mitotic index, and any combination thereof. Optionally, the negative indicator of senescence is a decrease in replicative capacity of the cells, a decrease in telomere length or a combination thereof. Optionally, the one or more candidate agents are selected from the group consisting of a peptide, nucleic acid, small molecule, and any combination thereof. Optionally, the method includes administering the candidate agent that promotes senescence to a subject. Optionally, the candidate agent that promotes senescence treats a cell proliferative disease or disorder in a subject.

As used herein, the term “positive indicator” refers to an marker, e.g., expression level or other parameter, increased or elevated as compared to a control. For example, a positive indicator of senescence is an indicator that is increased as compared a control, e.g., cells not undergoing or exhibiting signs of senescence. Positive indicators of senescence include, but are not limited to, an increase in lysosomal pH, an increase in protein aggregation, an increase in superoxide anion, an increase in expression of cell cycle inhibitors, an increase in expression of plasminogen activator inhibitor, an increase in senescence-associated beta-galactosidase positive cells, and any combination thereof.

As used herein, the term “negative indicator” refers to an indicator, e.g., expression level or other parameter, decreased as compared to a control. For example, a negative indicator of senescence is an indicator that is decreased as compared to a control, e.g., cells not undergoing or exhibiting signs of senescence. Negative indicators of senescence include, but are not limited to, a decrease in lysosomal enzyme activity, a decrease in nitric oxide levels, a decrease in nitrate levels, a decrease in activity of the NO synthase pathway, a decrease in cell proliferation, a decrease in angiogenic capacity, a change in morphology, a decrease in telomere length, reduced expression of the shelterin complex, a decrease in the mitotic index, and any combination thereof.

The terms higher, increased, elevated, or elevation refer to levels above a control or control level, e.g., an increase in an activity, response, condition, disease, or other biological parameter. For example, control levels are in vitro levels prior to, or in the absence of, addition of an agent or stimulus. This may include, for example, a 10% increase in the activity, response, condition, disease, or biological parameter as compared to the native or control level. Thus, the increase can be a 10, 20, 30, 40, 50, 60, 70, 80, 90, 100%, or any amount of increase in between as compared to native or control levels.

The terms low, lower, reduced, or reduction refer to any level below a control or control level, e.g., a decrease in an activity, response, condition, disease, or other biological parameter. For example, control levels are in vitro levels prior to, or in the absence of, addition of an agent or stimulus. This may include, for example, a 10% reduction in the activity, response, condition, disease, or biological parameter as compared to the native or control level. Thus, the reduction can be a 10, 20, 30, 40, 50, 60, 70, 80, 90, 100%, or any amount of reduction in between as compared to native or control levels.

A “control” sample or value refers to a sample that serves as a reference, usually a known reference, for comparison to a test sample. For example, a test sample, e.g., cultured cell line, can be taken under a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data using statistical analysis. For example, if values for a given parameter are widely variant in controls, variation in test samples will not be considered as significant.

One of skill in the art will understand which standard controls are most appropriate in a given situation and will be able to analyze data based on comparisons to standard control values. Standard controls are also valuable for determining the significance (e.g. statistical significance) of data. For example, if values for a given parameter are widely variant in standard controls, variation in test samples will not be considered as significant.

Indicators of senescence and methods for detecting indicators of senescence are known. See, e.g., Yepuri et al., Circulation Research, 2016 Jun. 10; 118 (12):e36-42; Yepuri et al., Aging cell, 11:1005-1016 (2012); Ghebremariam et al., PloS one, 8:e60653 (2013); Ramis et al., Biomedical microdevices, 15: 985-995 (2013); Rajapakse et al., PloS one, 6:e19237 (2011); Ramunas, et al., FASEB journal, 29:1930-1939 (2015); Fleenor et al., J. Vaasc. Res. 49:59-64 (2012), which are incorporated by reference herein in their entireties. The assay can be, for example, a RT-PCR assay, sequencing, or one of the provided methods described in the examples below.

Methods for detecting and identifying nucleic acids and proteins and interactions between such molecules involve conventional molecular biology, microbiology, and recombinant DNA techniques within the skill of the art. Such techniques are explained fully in the literature (see, e.g., Sambrook et al., Molecular Cloning: A Laboratory Manual, 3^{rd} Ed., Cold Spring Harbor Press, Cold Spring Harbor, N.Y. (2001); Animal Cell Culture, R. I. Freshney, ed., 1986).

Methods for detecting RNA are largely cumulative with the nucleic acid detection assays and include, for example, Northern blots, RT-PCR, arrays (including microarrays), and sequencing (including high-throughput sequencing methods). In some embodiments, a reverse transcriptase reaction is carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affymatrix. Optionally, methods for detecting RNA include sequencing methods. RNA sequencing are known and can be performed with a variety of platforms including, but not limited to, platforms provided by Illumina, Inc., (La Jolla, Calif.) or Life Technologies (Carlsbad, Calif.). See, e.g., Wang, et al., Nat Rev Genet. 10(1):57-63 (2009); and Martin, Nat Rev Genet. 12(10):671-82 (2011).

Various assays for determining levels and activities of proteins are available, such as amplification/expression methods, Western blotting, ELISA, ELISPOT, immunoprecipitation, immunofluorescence (e.g., FACS), immunohistochemistry, FISH, and shed antigen assays, southern blotting, sequencing, and the like. Moreover, the protein expression or amplification may be evaluated, e.g., by administering a molecule (such as an antibody) that binds the protein to be detected and is tagged with a detectable label (e.g. a radioactive isotope) and determining the location of the label. Thus, methods of measuring levels of protein levels in cells are generally known in the art and may be used to assess protein levels and/or activities in connection with the methods and compositions provided herein as applicable.

Binding assays are known and include, for example, a co-immunoprecipitation assay, a colocalization assay, or a fluorescence polarizing assay. The assays are known in the art, e.g., see Sambrook et al., Molecular Cloning: A Laboratory Manual, 3^{rd} Ed., Cold Spring Harbor Press, Cold Spring Harbor, N.Y. (2001); Dickson, Methods Mol. Biol. 461:735-44 (2008); Nickels, Methods 47(1):53-62 (2009); and Zinchuk et al., Acta Histochem. Cytochem. 40(4):101-11 (2007).

Any appropriate cell type or cell line can be used for analysis in the provided methods. Thus, the cells can be from (e.g., derived from) a biological sample. Biological sample or sample refers to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells), stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, and the like. Thus, the cells can be cells obtained from an organism, such as a mammal (e.g., a primate like a chimpanzee or human); cow; dog; cat; a rodent (e.g., guinea pig, rat, mouse); rabbit; bird; reptile; or fish. Optionally, the cells are cells of a cell line, optionally, obtained from, for example, the American Type Culture Collection (ATCC) (Manassas, Va.) or a commercial source.

Candidate agents suitable for use in the provided methods include, but are not limited to, antibodies, peptides, nucleic acids, small molecules and any combination thereof.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Optionally, the candidate agent is a nucleic acid, e.g., an inhibitory ribonucleic acid. Thus, optionally, the candidate agent is a functional nucleic acid. Such functional nucleic acids include, but are not limited to, antisense molecules and ribozymes. An inhibitory nucleic acid is a nucleic acid (e.g., DNA, RNA, and polymer of nucleotide analogs) that is capable of binding to a target nucleic acid (e.g., an mRNA translatable into a modulator of tumor immunosuppression) and reducing transcription of the target nucleic acid (e.g., mRNA from DNA) or reducing the translation of the target nucleic acid (e.g., mRNA) or altering transcript splicing (e.g., single stranded morpholino oligo). Optionally, the inhibitory nucleic acid is a nucleic acid that is capable of binding (e.g., hybridizing) to a target nucleic acid and reducing translation of the target nucleic acid. The target nucleic acid is or includes one or more target nucleic acid sequences to which the inhibitory nucleic acid binds (e.g., hybridizes). Thus, an inhibitory nucleic acid typically is or includes a sequence (also referred to as an antisense nucleic acid sequence) that is capable of hybridizing to at least a portion of a target nucleic acid. An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. The inhibitory nucleic acid can be single or double stranded. The use of inhibitory methods to inhibit the in vitro translation of genes is well known in the art (Marcus-Sakura, Anal. Biochem., 172:289, (1988)).

The term polypeptide, as used herein, generally has its art-recognized meaning of a polymer of at least three amino acids and is intended to include peptides and proteins. However, the term is also used to refer to specific functional classes of polypeptides, such as, for example, desaturases, elongases, etc. For each such class, the present disclosure provides several examples of known sequences of such polypeptides. Those of ordinary skill in the art will appreciate, however, that the term polypeptide is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those in the art understand that protein sequences generally tolerate some substitution without destroying activity.

As used herein, the term antibody refers to an immunoglobulin. Whenever the term antibody is used, however, a functional fragment of an antibody can be used. The antibody or fragment may be of any type (e.g., IgG, IgA, IgM, IgE or IgD). Preferably, the antibody is IgG. An antibody may be non-human (e.g., from mouse, goat, or any other animal), fully human, humanized, or chimeric. An antibody may be polyclonal or monoclonal. Optionally, the antibody is monoclonal. The term monoclonal antibody as used herein, refers to a pure, target-specific antibody produced from a single clone of cells grown in culture and that is capable of indefinitely proliferating. Monoclonal antibodies that may be used include naked antibodies, that attach to and block antigens on cancerous cells. Antibodies that may be used in the provided method include conjugated antibodies, such as tagged, labeled, or loaded antibodies. Specifically, the antibodies may be tagged or loaded with a drug or a toxin, or radioactively labeled.

As used herein, the term antibody fragment refers to any portion of the antibody that recognizes an epitope. Antibody fragments may be glycosylated. By way of non-limiting example, the antibody fragment may be a Fab fragment, a Fab′ fragment, a F(ab′)2 fragment, a Fv fragment, an rIgG fragment, a functional antibody fragment, single chain recombinant forms of the foregoing, and the like. F(ab′)2, Fab, Fab′ and Fv are antigen-binding fragments that can be generated from the variable region of IgG and IgM. They vary in size, valency, and Fc content. The fragments may be generated by any method, including expression of the constituents (e.g., heavy and light chain portions) by a cell or cell line, or multiple cells or cell lines. Preferably, the antibody fragment recognizes the epitope and contains a sufficient portion of an Fc region such that it is capable of binding an Fc receptor.

As noted above, the provided methods optionally include administering to the subject a candidate agent that inhibits senescence. Such agents can be used to prevent or treat an age-related disease or disorder. Optionally, the age-related disease is associated with the cell cycle, mitochondrial biogenesis, oxidative stress, or telomere dysfunction. Such agents include, but are not limited to, anti-aging agents such as, for example, inhibitors of reactive oxygen species, modulators of mitochondrial activity or biogenesis, and modulators of telomere length. Candidate agents that inhibit senescence can be small molecules, antibodies, peptides, proteins, DNAs, RNAs, or metabolic intermediates thereof. Thus, the candidate agent that inhibits senescence can, for example, result in increased mitochondrial biogenesis and function, reduced ROS levels, or extended life span of senescent cells and post-mitotic cells such as neuron cells.

Optionally, the provided method include administering to the subject a candidate agent that promotes senescence. Such agents can be used, for example, to prevent or treat a cell proliferative disease or disorder, which include any cellular disorder in which the cells proliferate more rapidly than normal tissue growth. Thus a “proliferating cell” is a cell that is proliferating more rapidly than normal cells. Candidate agents that inhibit senescence can be small molecules, antibodies, peptides, proteins, DNAs, RNAs, or metabolic intermediates thereof. Candidate agents that promote senscence can be used to treat proliferative diseases including cancer, bone disorder, inflammatory disease, immune disease, nervous system disease, metabolic disease, respiratory disease, thrombosis, or cardiac disease or any other disorder associated with abnormal cell proliferation.

Also provided herein are methods of promoting senescence of a proliferative cell comprising contacting the proliferative cell with a composition comprising an effective amount of one or more proton pump inhibitors, wherein the proliferative cell is not a tumor cell. Optionally, the proliferative cell is not a solid tumor cell. Optionally, the proliferative cell is located in a subject. Thus, also provided are methods of promoting senescence of a proliferative cell in a subject comprising administering to the subject a composition comprising an effective amount of one or more proton pump inhibitors, wherein the proliferative cell is not a tumor cell. Optionally, the tumor cell is not a solid tumor cell. Optionally, the proliferative cell is a a skin cell or a vascular cell. Optionally, the proliferative cell is a enodthelial cell, keratinocyte, or fibroblast cell. Optionally, the composition is formulated for topical administration. Optionally, the composition is formulation for ocular, oral, inhalation, intravenous, intrathecal, intra-uterine, intraperitoneal, intravesical, intra-articular, intramuscular or subcutaneous administration. Optionally, the composition comprises 1 to 20 μm of the one or more proton pump inhibitors. Optionally, the subject has a proliferative disease or disorder. Optionally, proliferative disease or disorder is bone disorder, inflammatory disease, immune disease, nervous system disease, metabolic disease, respiratory disease, thrombosis, or cardiac disease or any other disorder associated with abnormal cell proliferation. Optionally, the proliferative disease is a disease characterized by hyperplasia. Thus, provided herein are methods of promoting senescence of a proliferative cell in a subject with a disease characterized by hyperplasia comprising contacting the proliferative cell with a composition comprising an effective amount of one or more proton pump inhibitors, wherein admininstration promotes senescence of the proliferative cell and treats the disease characterized by hyperplasia in the subject. As used herein, hyperplasia refers to an increase in amount of an organic tissue resulting from cell proliferation. During hyperplasia, increased nutrition is needed to support cell proliferation. This demand for increased nutrition is associated with an increased ingrowth of blood vessels. The growth of such hyperplastic tissue would be reduced or blocked by agents that accelerate endothelial aging, such as the PPIs. Therefore PPIs, by accelerated endothelial senescence, will slow or block the growth of hyperplastic tissue. Diseases characterized by hyperplasia include, but are not limited to, myointimal hyperplasia (that causes narrowing of blood vessels and bypass grafts in patients that are treated with angioplasty or stenting or bypass surgery for peripheral or coronary artery disease); keloid disorder (in patients that have an injury or a surgically-induced incision); vascular malformations; intestinal polyps; prostatic hyperplasia; endometrial hyperplasia; eczema and psoriasis. Optionally, the proliferative disease or disorder is a keloid disorder or myointimal hyperplasia.

In an alternative embodiment, the proliferative cell can be a tumor cell. Thus, provided are methods of promoting senescence of a tumor cell in a subject comprising administering to the subject a composition comprising an effective amount of one or more proton pump inhibitors. Optionally, the composition is formulation for topical, ocular, oral, inhalation, intravenous, intrathecal, intra-uterine, intraperitoneal, intravesical, intra-articular, intramuscular or subcutaneous administration. Optionally, the composition comprises 1 to 20 μm of the one or more proton pump inhibitors.

Provided herein are compositions comprising the proton pump inhibitors. Also provided are compositions comprising one or more of the candidate agents identified by the provided methods. Optionally, the compositions comprise a candidate agent identified by the provided methods as inhibiting senescence. Optionally, the compositions comprise a candidate agent identified by the provided methods as promoting senescence. Optionally, the compositions comprise a pharmaceutically acceptable excipient or pharmaceutically acceptable carrier. Suitable carriers and their formulations are described in Remington: The Science and Practice of Pharmacy, 22nd Edition, Loyd V. Allen et al., editors, Pharmaceutical Press (2012). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

For topical administration, the compounds can be formulated as solutions, gels, ointments, creams, suspensions, etc. as are well-known in the art. Systemic formulations include those designed for administration by injection, e.g., subcutaneous, intravenous, intramuscular, intranasal, intrathecal or intraperitoneal injection, as well as those designed for transdermal, transmucosal, oral or pulmonary administration.

For injection, the compounds can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, the compounds can be in powder form for constitution with a suitable vehicle, e.g., sterile pyrogen-free water, before use.

For oral administration, the compounds can be readily formulated by combining the active peptides (or antibodies) or peptide analogues (or antibody fragments) with pharmaceutically acceptable carriers well known in the art. Such carriers enable the compounds of the invention to be formulated as tablets, pills, draggers, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, and the like. Additionally, flavoring agents, preservatives, coloring agents and the like may be added. For buccal administration, the compounds may take the form of tablets, lozenges, and the like. formulated in conventional manner.

For administration by inhalation, the compounds are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also maybe employed, although usually at the cost of greater toxicity. Additionally, the compounds may be delivered using a sustained-release system, such as semi-permeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

According to the methods provided herein, the subject is administered an effective amount of one or more of the agents provided herein. The terms effective amount and effective dosage are used interchangeably. The term effective amount is defined as any amount necessary to produce a desired physiologic response (e.g., induction of senescence). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous, or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly, or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Provided herein are kits comprising a mammalian cell line and one or more proton pump inhibitors. Optionally, the one or more proton pump inhibitors are selected from the group consisting of lansoprazole, dexlansoprazole, omeprazole, esomeprazole, pantoprazole, rabeprazole, and ilaprazole. Optionally, the kit further comprises one or more reagents for assaying an indicator of senescence. For example, the kit can include, primers, probes, labels, antibodies or other reagents capable for assaying an indicator of senescence. Optionally, the mammalian cell line is a primary cell line or immortalized cell line. Optionally, the mammalian cell line is selected from the group consisting of an endothealial cell line, a fibroblast cell line or a keratinocyte cell line. Optionally, the mammalian cell line is selected from the group consisting of human umbilical venous endothelial cells, human aortic endothelial cells, human coronary artery endothelial cells, human microvascular endothelial cells. Optionally, the kit further comprises reagents for inducing senescence. Optionally, the kit further comprises instructions for use.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims.

### EXAMPLES

### Example 1. Proton Pump Inhibitors Accelerate Endothelial Senescence

In low-pH conditions of the gastric parietal cell, proton pump inhibitors (PPIs) are converted to the active sulfenic acid form. When activated, the PPIs form a mixed disulfide with the proton pump of the parietal cell to inhibit its secretion of HCl into the stomach. Physicians have prescribed these drugs with the perception that these agents have specificity for the parietal cells of the stomach. However, similar proton pumps are also found in cell lysosomes. An earlier publication found no evidence that the PPI rabeprazole impaired lysosomal activity in hepatic cells (Fujisaki H, et al., Jpn J Pharmacol. 1998; 76:279-288). However, PPIs may also affect endothelial lysosomes and disrupt proteostasis.

The following experiments were performed to study the long-term effect of PPIs on endothelial dysfunction and senescence and investigate the mechanism involved in PPI-induced vascular dysfunction. As described in more detail below, chronic exposure to PPIs impaired endothelial function and accelerated human endothelial senescence by reducing telomere length. These data provide a unifying mechanism for the association of PPI use with increased risk of cardiovascular, renal, and neurological morbidity and mortality.

### Materials and Methods

Cell culture. Human microvascular endothelial cells (ECs) purchased from Lonza (cat # CC-2543, Basel, Switzerland) were cultured continuously for three passages from passage (P) 4 to P6 in the presence of clinically relevant doses of esomeprazole or vehicle (DMSO) (Shin J M, Kim N. Pharmacokinetics and pharmacodynamics of the proton pump inhibitors. Journal of neurogastroenterology and motility. 2013; 19:25-35). All in vitro experiments were performed at P6 upon confluency. ECs were cultured and maintained in EBM-2 Basal Medium (cat # CC-3156, Lonza, Basel, Switzerland) supplemented with EGM-2 MV SingleQuots™ Kit—growth factors, cytokines, and antibiotics (cat # CC-4147, Lonza, Basel, Switzerland).

Lysosomal studies. Cellular pH, lysosomal cathepsin B and protein aggregation were measured in live ECs using pHrodo™ Green/Red dextran, 10,000 MW dye (cat: P35368/P35372, MOLECULAR PROBES®, Life Technologies, Carlsbad, Calif.), Magic Red™ Cathepsin B Assay Kit (cat: 938, ImmunoChemistry Technologies LLC, Bloomington, Minn.) and PROTEOSTAT® Protein aggregation assay (cat: ENZ-51023-KP002, Enzo Life Sciences, Inc., Farmingdale, N.Y.). The pHrodo™ Green dextran when endocytosed permits visualization of endosomal pH, with an inverse non-linear relationship of fluorescence intensity to pH. The studies were confirmed with pHrodo™ Red dextran which provided qualitatively similar findings. All experiments were performed according to manufacturer's user guide. Fluorescence images were taken using Olympus IX51 Inverted fluorescence microscope at 10× magnification and 40× for protein aggregation. All images were quantified using NIH ImageJ 1.47v software. Acid phosphate and N-Acetyl-β-D-Glucosaminidase activity were measured using kits from Sigma-Aldrich, Inc. (cat: CS0740 and CS0780, St. Louis, Mo.). Cells were harvested using CelLytic™ M (cat: C2978, Sigma-Aldrich, Inc., St. Louis, Mo.); fresh cell lysate was used for all experiments. Assay and interpretation of results were performed using manufacturer's instructions. Absorbance was measured using Tecan Infinite® M1000 PRO multimode reader at 405 nm (Tecan, Mannedorf, Switzerland).

Measurement of superoxide and nitric oxide. Superoxide and nitric oxide were measured in ECs using live cell imaging dyes; DHE (Molecular Probes® cat # D-1168, Eugene, Oreg.) for superoxide and DAF-2DA (Sigma cat #50277, St. Louis, Mo.) for nitric oxide measurement according to the published protocol (Yepuri G, et al., Positive crosstalk between arginase-ii and s6k1 in vascular endothelial inflammation and aging. Aging cell. 2012; 11:1005-1016). Several images per well were captured using Olympus IX51 Inverted fluorescence microscope. Relative fluorescence intensities of images were quantified using NIH ImageJ software. The total NO concentration (NOx) was determined using the Griess colorimetric assay as previously described (Ghebremariam Y T, et al., Fxr agonist int-747 upregulates ddah expression and enhances insulin sensitivity in high-salt fed dahl rats. PloS one. 2013; 8:e60653).

Assessment of cell proliferation by RTCA and BrdU assay. Cell proliferation was assessed using two different approaches. In a first approach, EC proliferation was measured by measuring cell index (CI; an impedance measurement correlated with cell proliferation) using the instrument xCELLigence Real-Time Cell Analyzer (RTCA) (Ramis G, et al., Optimization of cytotoxicity assay by real-time, impedance-based cell analysis. Biomedical microdevices. 2013; 15:985-995) developed by ACEA Biosciences, Inc. (San Diego, Calif.). Experiments were performed according to manufacturer's instructions. In brief, confluent ECs that had been treated with esomeprazole or vehicle for three passages were dissociated using TrypLE™ Express (Gibco® by Life Technologies cat #12605, Carlsbad, Calif.). 10000 cells/well were plated and maintained for 115-120 hours in the xCELLigence RTCA. Later, the CI values were plotted and represented as area under the curve. In a second approach, cell proliferation was assessed by using the CytoSelect™ BrdU Cell Proliferation ELISA Kit from Cell Biolabs, Inc. (Cat: CBA-251, San Diego, Calif.). The protocol was performed according to manufacturer's instructions. Colorimetric detection of signal was obtained using Tecan Infinite® M1000 PRO multimode reader at absorbance wavelength 450 nm.

Network formation assay to measure angiogenic capacity. In order to assess angiogenic capacity of ECs, growth factor reduced matrigel from BD Biosciences (cat #354230, San Jose, Calif.) was used. In brief, 24 well flat transparent plates were coated with 200 μl of matrigel/well and allowed to set at 37° C. for one hour. Subsequently, 1×10⁵ cells/well were seeded and allowed to incubate for 16 hours at 37° C. Several images per well were obtained using Olympus IX51 Inverted fluorescence and bright field microscope. Network branching was quantified using NIH ImageJ software. Total length is equal to the summed length of all segments, branches and isolated branches not within the main network. Total branching length equals the summed length of total segments and total branches within the main network. Total segment length is the summed length of all the segments within the network without branching length.

Senescence-associated β-galactosidase (SA-β-gal) staining. Senescence of ECs was measured using the Cellular Senescence Assay kit from Cell Biolabs, Inc. (Cat: CBA-230, San Diego, Calif.). The protocol was performed according to the manufacturer's guide. Briefly, Ecs treated continuously for 3 passages with esomeprazole or vehicle were plated in a 6 well plate. Upon confluency, cells were initially fixed and later incubated with SA-β-gal working solution for 16 hours in a non-humidified CO₂ free incubator at 37° C. After incubation the cells were counter stained with SYTO® GREEN fluorescent nucleic acid stain (Life Technologies). Several random images per well were taken using light and fluorescent microscope. The SA-β-gal positive cells were counted manually and total cell number per field was quantified using NIH ImageJ software.

Long term studies of endothelial histology. After ECs were exposed to vehicle or ESO (5 or 10 uM) through 3 passages (P4-P6), the treatment with ESO or vehicle was discontinued when the cells reached confluency at P6. Subsequently the cells in all groups were maintained in endothelial growth medium which was routinely replaced with fresh medium every 48 hours for 81 days. Microphotographs were taken at regular intervals throughout, and on the final day of culture.

Western blot analysis. Preparation of cell lysate, SDS-PAGE, transfer of proteins onto membrane and western blotting was performed as described previously (Rajapakse A G, et al., Hyperactive s6k1 mediates oxidative stress and endothelial dysfunction in aging: Inhibition by resveratrol. PloS one. 2011; 6:e19237.6). To detect PAI-1 protein expression anti-PAI-1 rabbit monoclonal antibody (Cell Signaling Technology, Inc. cat #11907S, Danvers, Mass.) was used and anti-α-Tubulin mouse monoclonal antibody (Sigma cat # T5168, St. Louis, Mo.) was used to normalize expression.

Quantitative PCR and PCR array. Total RNA was isolated from cultured cells using PerfectPure RNA Cultured Cell Kit-50 from 5 PRIME (cat #2900319, San Francisco, Calif.) according to the manufacturer's instruction. Complementary DNA (cDNA) was prepared using gScript™ cDNA SuperMix (Quanta BioSciences, Inc. cat #95048, Beverly, Mass.). Quantitative PCR (qPCR) was performed using Taqman gene expression assays (Applied Biosystems, Foster City, Calif.). All genes analyzed for expression (listed in the table below) were normalized to GAPDH expression and expressed as relative fold changes using the ΔCt method of analysis. Gene expression of shelterin complex genes was quantified using SYBR™ Green Real Time PCR master mix. Primers for genes related to shelterin complex were obtained from Integrated DNA Technologies, Inc. (San Jose, Calif.) and are listed in the table below. RT² Profiler PCR Array (Qiagen, Hilden, Germany) was used to assess expression of selected genes associated with specific cellular functions following manufacturer's instructions. Name, catalog numbers and genes detected by the arrays are listed below.

TABLE 1
Taqman genes (human) and catalog number used for qPCR

| SL # | Gene Name | Catalog # |
|---|---|---|
| 1 | SERPINE1/PAI-1 | Hs01126606_m1 |
| 2 | CDKN1A(p21) | Hs00355782_m1 |
| 3 | COL1A1 | Hs00164004_m1 |
| 4 | vWF | Hs01109446_m1 |
| 5 | DDAH1 | Hs00201707_m1 |
| 6 | DDAH2 | Hs00967863_g1 |
| 7 | eNOS | Hs01574659_m1 |
| 8 | iNOS | Hs01075529_m1 |
| 9 | SMAD3 | Hs00969210_m1 |
| 10 | Twist1 | Hs00361186_m1 |
| 11 | GAPDH | Hs02758991_g1 |

TABLE 2
Primers and sequence information for shelterin complex genes

| SL# | Gene | Sense | Antisense |
|---|---|---|---|
| 1 | TRF1 | TCTGCGGTAACTGAATCCTC | GTTACCGGCTGACTCTTTGA |
| | | (SEQ ID NO: 1) | (SEQ ID NO: 2) |
| 2 | TRF2 | AGACTTGGGTGGAAGAGGA | TAATCATCACAGCTGTTCGG |
| | | (SEQ ID NO: 3) | (SEQ ID NO: 4) |
| 3 | POT1 | TGTGGCAAGATCTCTGAAGG | TCTGAATGCTGATTGGCTGT |
| | | (SEQ ID NO: 5) | (SEQ ID NO: 6) |
| 4 | TPP1 | GGGAGGACCAGGAGCAT | GGGCCTAGAGAGCTCAGAAT |
| | | (SEQ ID NO: 7) | (SEQ ID NO: 8) |
| 5 | TIN2 | TTGCCTGGAGACAATATGGT | GTCGGCCAGCTAGAGGTT |
| | | (SEQ ID NO: 9) | (SEQ ID NO: 10) |
| 6 | RAP1 | GCCACCCGGGAGTTTGA | GGGTGGATCATCATCACACAT |
| | | (SEQ ID NO: 11) | (SEQ ID NO: 12) |

TABLE 3
RT Profiler PCR Array (human) QIAGEN

| SL # | Pathway | Catalog # |
|---|---|---|
| 1 | Cellular Senescence | PAHS-050Z |
| 2 | Epithelial-Mesenchymal Transition | PAHS-090Z |
| 3 | TGFB BMP Signaling Pathway | PAHS-035Z |
| 4 | Angiogenesis | PAHS-024Z |
| 5 | Endothelial Cell Biology | PAHS-015Z |

Telomere length and telomerase activity. Telomere length in ECs was measured using monochrome multiplex qPCR (MMqPCR) assay as described previously (Ramunas J, et al., Transient delivery of modified mrna encoding tert rapidly extends telomeres in human cells. FASEB journal: official publication of the Federation of American Societies for Experimental Biology. 2015; 29:1930-1939). The telomeric repeat amplification protocol (TRAP) was performed using TRAPeze® Telomerase Detection Kit (EMD Millipore Inc. cat # S7700, Darmstadt, Germany). Experiment was performed according to the manufacturer's instructions. In brief, ECs treated with esomeprazole or vehicle were collected, counted and total protein was isolated from 100,000 cells. The amount of lysate used per reaction was normalized to the amount of 1,000 cells for ECs and 500 cells for telomerase positive control (PC). In short, TRAP was performed at 30° C. for 30 minutes and resulting products were amplified in a 29 cycle PCR reaction. Heat inactivated (hi) lysate samples for each condition were used as internal negative controls.

Statistical analysis. All data, unless stated otherwise, was analyzed using GraphPad PRISM 6 software (GraphPad, La Jolla, Calif.). n represents average of 2-3 technical replicates. One-way ANOVA was used for multiple comparisons followed by Bonferroni posthoc correction. The differences between vehicle and treatment groups in each subgroup was analyzed using unpaired t test. All data is expressed as mean±SEM. Group differences were considered statistically significant at p<0.05.

### Results

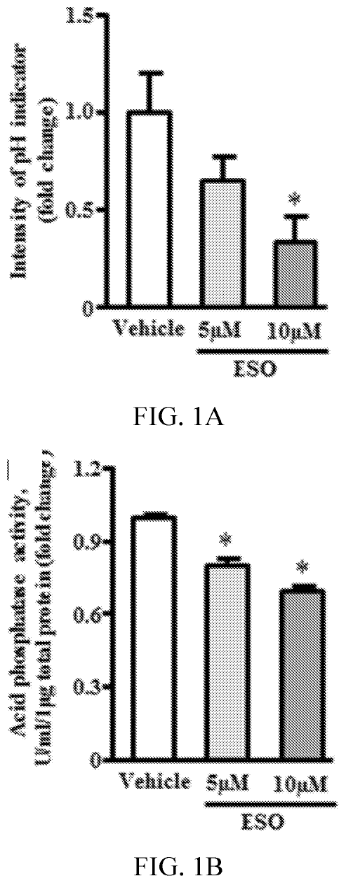

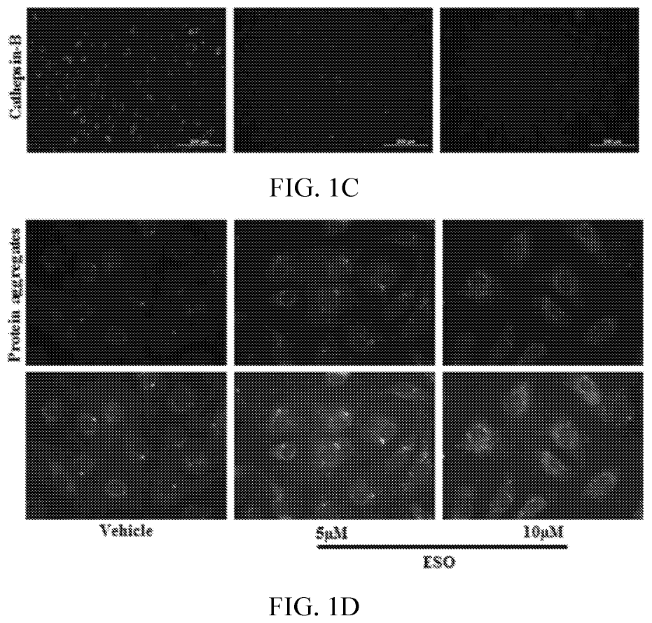

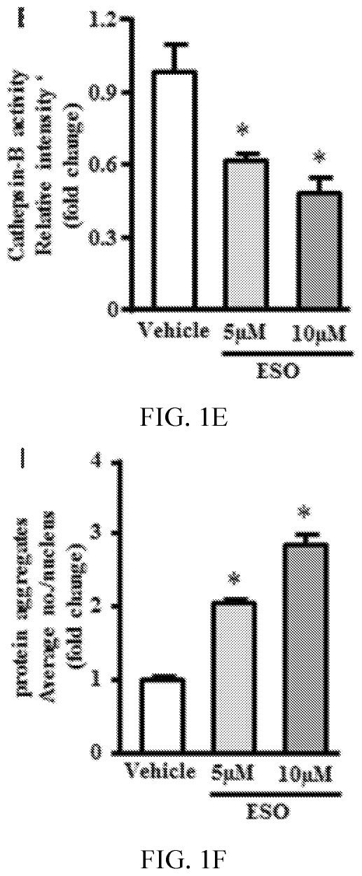

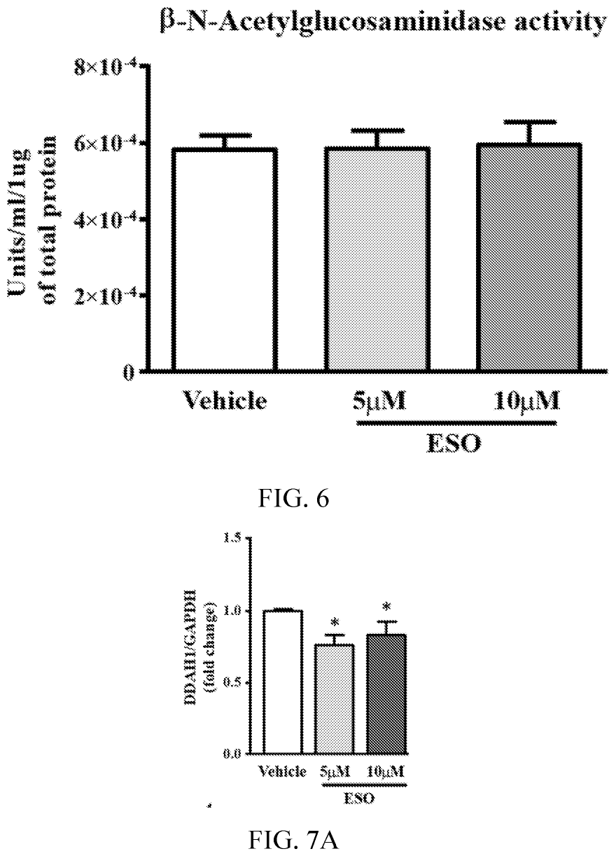

The PPI esomeprazole impairs human lysosomal function and proteostasis. Human microvascular ECs were cultured continuously for 3 passages (passages 4-6) in media containing a clinically relevant concentration of the PPI esomeprazole (5 and 10 μmol/L) or vehicle (DMSO). Using a pH-sensitive fluorescent dye that is taken up by endocytosis, fluorescence was observed in a perinuclear distribution consistent with lysosomal localization in EC treated with vehicle. In ECs chronically exposed to esomeprazole, fluorescence intensity was significantly reduced, consistent with an increase in lysosomal pH (FIG. 1A). These studies were repeated using a second pH-sensitive fluorescent dye and obtained qualitatively similar findings (FIG. 5). An impairment in the lysosomal proton pump and an increase in lysosomal pH would be expected to impair lysosomal enzymes, which are optimally active at a pH of ≈4.80 (Ohkuma S, Poole B. Fluorescence probe measurement of the intralysosomal pH in living cells and the perturbation of pH by various agents. Proc Natl Acad Sci USA. 1978; 75:3327-3331; Liu W, et al., Inhibition of lysosomal enzyme activities by proton pump inhibitors. J Gastroenterol. 2013; 48:1343-1352. doi: 10.1007/s00535-013-0774-5). Indeed, the activity of lysosomal cathepsin-B and acid phosphatase was reduced in ECs treated chronically with esomeprazole (FIGS. 1B, 1C, and 1E). No difference was observed in N-acetyl-β-d-glucosaminidase activity (FIG. 6). Using a commercially available protein aggregation detection dye, together with image quantification software to quantify protein aggregates, an increase in protein aggregates was observed in the esomeprazole-treated ECs (FIGS. 1D and 1F). These studies indicate that PPIs impair endothelial lysosomal acidification, enzyme activity, and proteostasis.

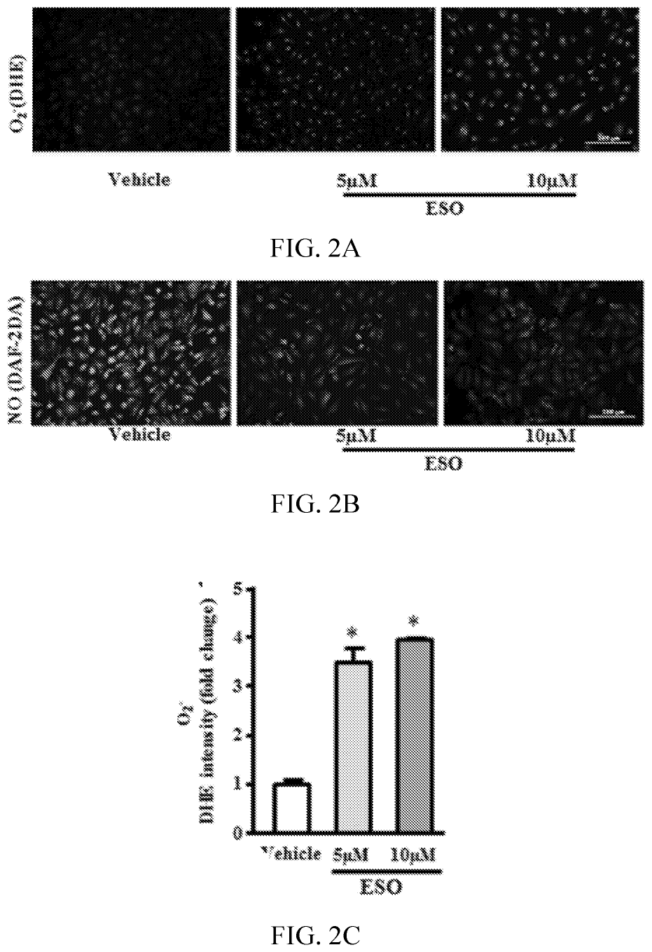

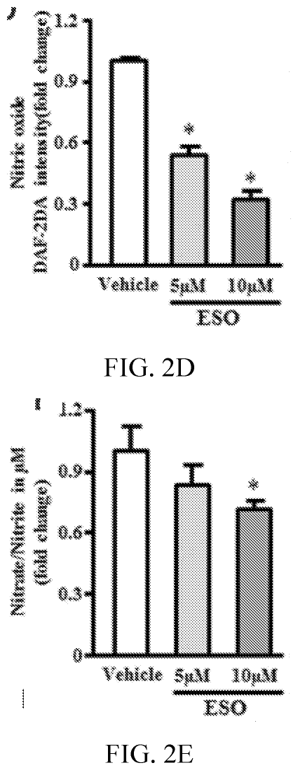

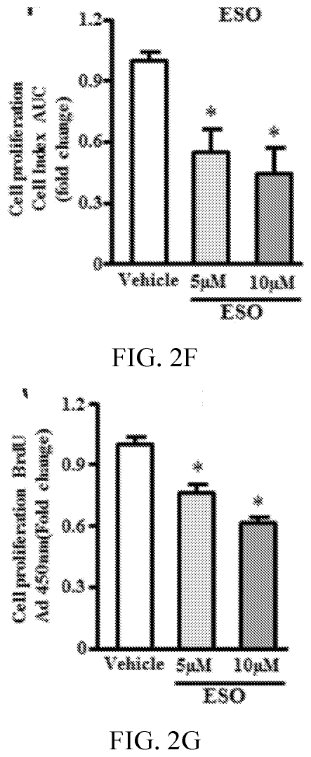

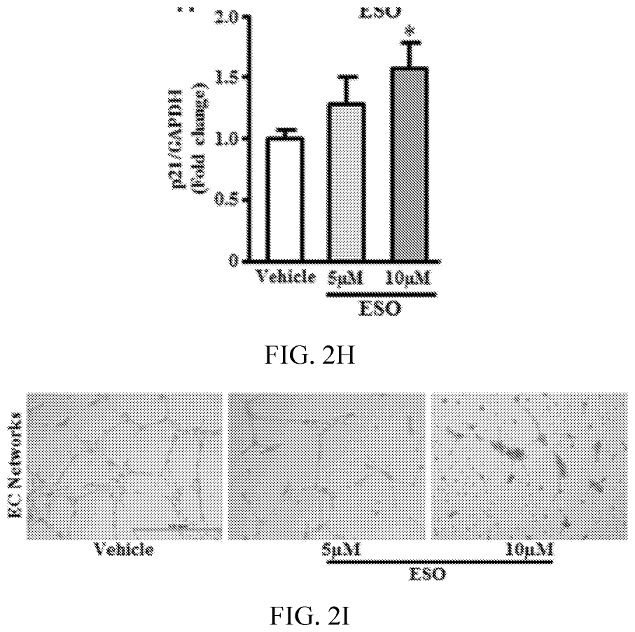

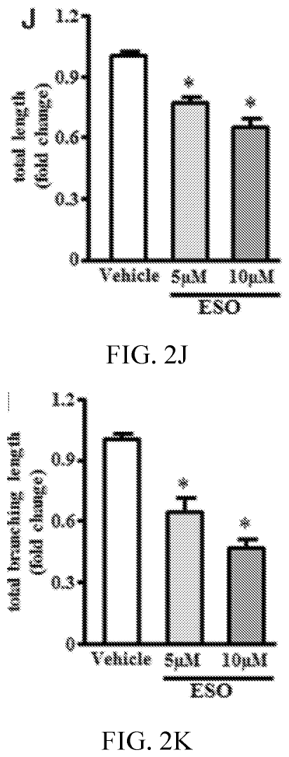

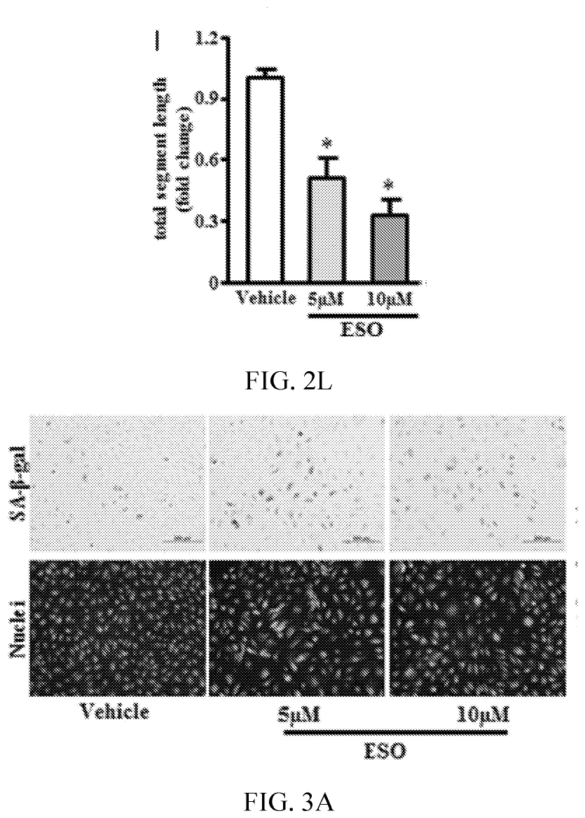

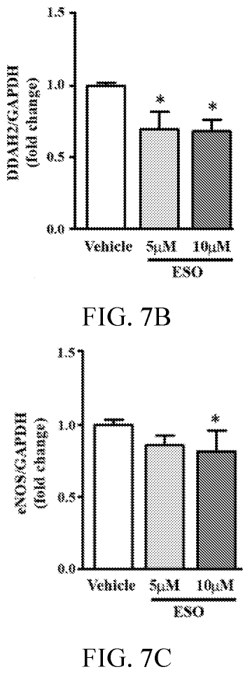

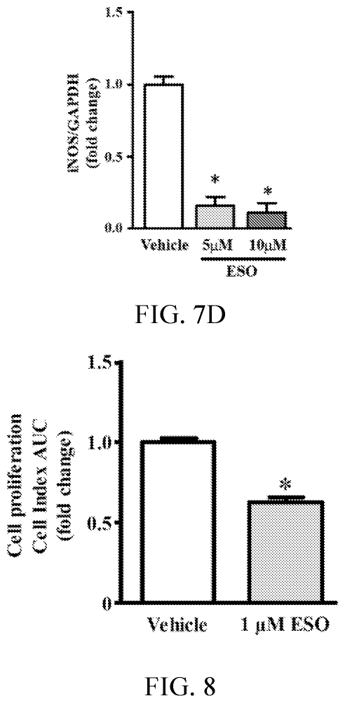

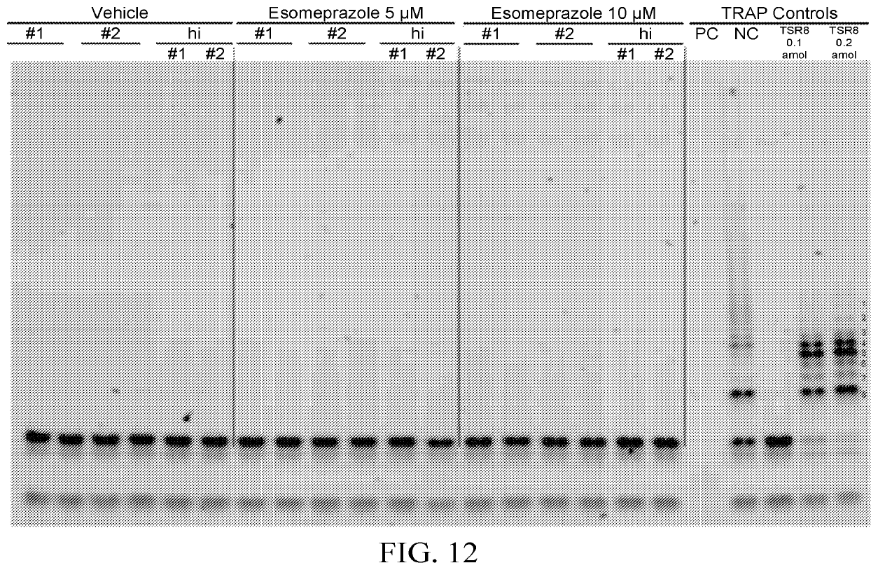

Disruption of proteostasis is associated with a global deterioration of cell function and accelerated cell aging (Balch W E, et al., Adapting proteostasis for disease intervention. Science. 2008; 319:916-919. doi: 10.1126/science.1141448; Ben-Zvi A, et al., Collapse of proteostasis represents an early molecular event in Caenorhabditis elegans aging. Proc Natl Acad Sci USA. 2009; 106:14914-14919. doi: 10.1073/pnas.0902882106; Chondrogianni N, Fragoulis E G, Gonos E S. Protein degradation during aging: the lysosome-, the calpain- and the proteasome-dependent cellular proteolytic systems. Biogerontology. 2002; 3:121-123). A hallmark of endothelial dysfunction is an increase in the generation of superoxide anion (Harrison D G. Cellular and molecular mechanisms of endothelial cell dysfunction. J Clin Invest. 1997; 100:2153-2157. doi: 10.1172/JCI119751; Rajapakse A G, et al., Hyperactive S6K1 mediates oxidative stress and endothelial dysfunction in aging: inhibition by resveratrol. PLoS One. 2011; 6:e19237. doi: 10.1371/journal.pone.0019237) and a decrease in nitric oxide (NO) levels (Cooke J P, Dzau V J. Derangements of the nitric oxide synthase pathway, L-arginine, and cardiovascular diseases. Circulation. 1997; 96:379-382). Using fluorescent live cell imaging dyes, it was observed that by comparison with EC treated with vehicle, those treated chronically with esomeprazole produced more superoxide anion as measured by dihydroethidium and generated less NO as measured by diamino fluorescein 2-diacetate staining. This impairment in EC function was confirmed by a decrease in total nitrate levels as detected by Griess colorimetric assay (FIG. 2A-2E) in the esomeprazole-treated group. Also observed was a decrease in the expression of DDAH1/2 (dimethylarginine dimethylaminohydrolase, isoforms 1 or 2), eNOS (endothelial nitric oxide synthase), and iNOS (inducible nitric oxide synthase) (FIG. 7); a reduced expression of these critical enzymes in the NO synthase pathway would explain a decline in EC NO generation. Because NO plays a key role in EC proliferation and angiogenesis (Cooke J P, Losordo D W. Nitric oxide and angiogenesis. Circulation. 2002; 105:2133-2135. doi: 10.1161/01.CIR.0000014928.45119.73), these EC functions were assessed. Chronic exposure to esomeprazole dose-dependently impaired cell proliferation as measured by 5-bromo-2′-deoxyuridine assay (FIG. 2F), a finding which was confirmed using a realtime cell analyzer, which assesses cell growth (FIG. 2G). Additional studies revealed that chronic exposure (3 passages) to a concentration of esomeprazole as low as 1 μmol/L significantly reduced EC proliferation as measured by real-time cell analyzer (FIG. 12). Consistent with these observations, it was observed that chronic esomeprazole treatment increased the expression of cell cycle inhibitor p21 gene (FIG. 2H). Finally, it was noted that esomeprazole impaired the angiogenic capacity of ECs as measured by network formation on growth factor-depleted matrigel (FIG. 2I-2L). These results indicate that esomeprazole impairs multiple endothelial functions.

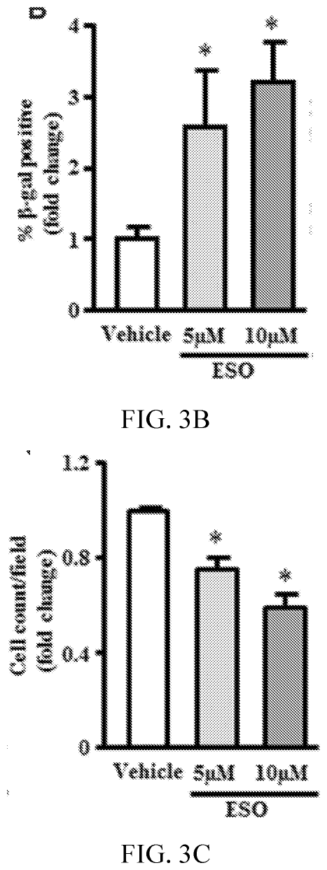

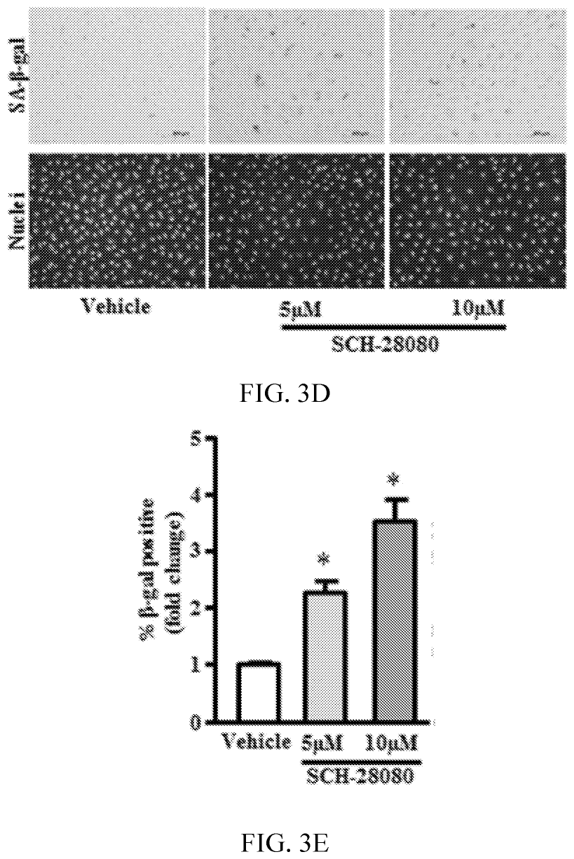

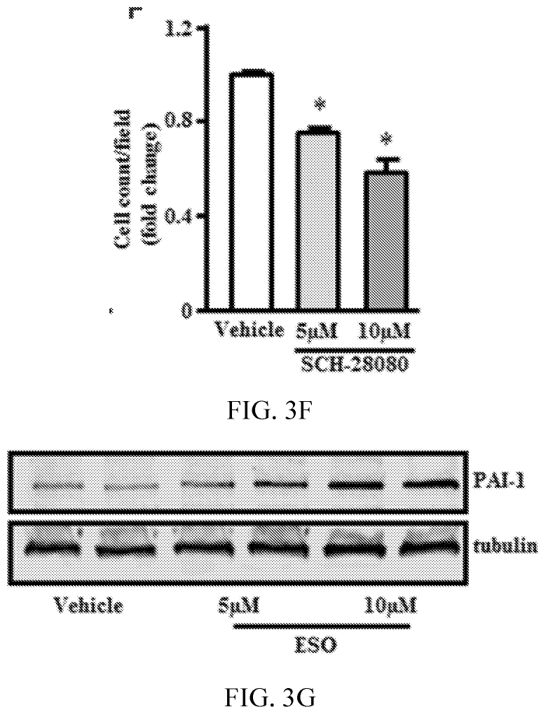

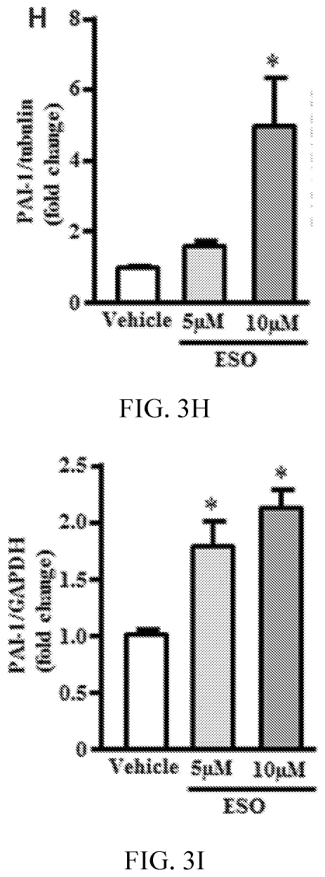

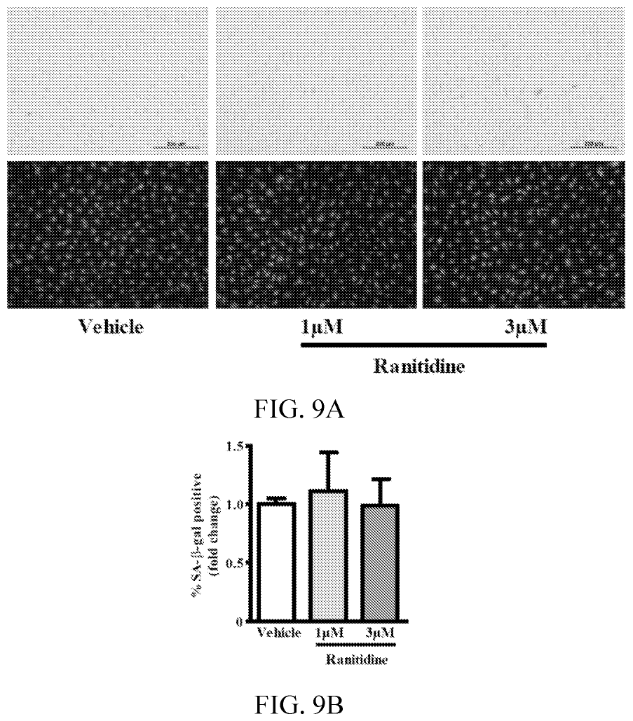

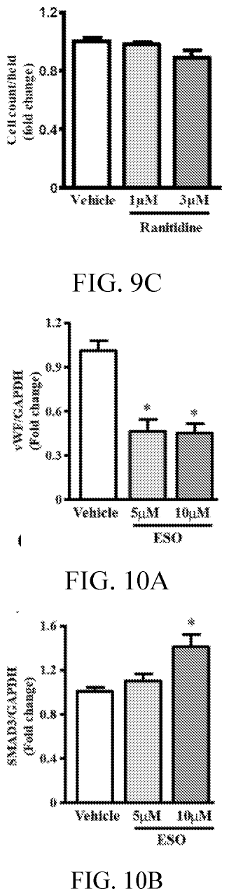

Impairment of proteostasis and reduced cell proliferation are hallmarks of cellular senescence (Chondrogianni N, Fragoulis E G, Gonos E S. Protein degradation during aging: the lysosome-, the calpain- and the proteasome-dependent cellular proteolytic systems. Biogerontology. 2002; 3:121-123; Lahteenvuo J, Rosenzweig A. Effects of aging on angiogenesis. Circ Res. 2012; 110:1252-1264. doi: 10.1161/CIRCRESAHA.111.246116). To determine if cells chronically treated with PPIs exhibited other features of senescence, the effect of chronic treatment with esomeprazole or with SCH-28080 (another H+K+ATPase inhibitor with a potency similar to omeprazole, IC50 of 2.5 and 4.0 μmol/L, respectively) was assessed. It was found that senescence-associated β-galactosidase (SA-β-gal)-positive cells were increased by comparison to vehicle (FIGS. 3A, 3B, 3D, and 3E) as early as P6 in both esomeprazole- and SCH-28080-treated groups. Also, observed was a decrease in total cell count per microscopic field (FIGS. 3E and 3F) by SYTO-green staining consistent with a decline in cell proliferation. Also noted was a change in the morphology in some of the PPI-treated cells; some of which adopted the friedegg morphology characteristic of senescent EC. Interestingly, not observed was any significant difference in SA-β-gal-positive cell or total cell count on treatment with ranitidine (FIG. 9A-9C; ranitidine is a H2 histamine receptor antagonist, which is used as an alternative treatment for gastroesophageal reflux disease). Further investigated was the expression of 331 genes from 5 different molecular pathways (cellular senescence, EC biology, angiogenesis, transforming growth factor-β-bone morphogenic protein, and epithelial to mesenchymal transition signaling pathways) involved in esomeprazole-induced endothelial dysfunction using polymerase chain reaction array. It was observed that 52 genes were upregulated (>2-fold increase) and 49 genes were downregulated (>0.5-fold of control value). In general, the changes in gene expression were consistent with those observed in endothelial senescence, for example, increased expression of genes involved in endothelial-to-mesenchymal transition (EndoMT), inflammation, and increased oxidative stress (Tables 4 and 5).

TABLE 4
Expressin of genes (PCR array) that are strongly up-regulated (>2 fold) upon
treatment with esomeprazole compared to vehicle (DMSO).

| Gene | | |
|---|---|---|
| symbol | Gene name | Function |
| AGTR1 | Angiotensin II | Vasocontriction |
| | receptor type I | |
| ALDH1A3 | Aldehyde | Detoxification of aldehydes produced during |
| | dehydrogenase 1 | lipid peroxidation and alcohol metabolism |
| | family member A3 | |
| ANGPT1 | angiopoietin 1 | secreted glycoprotein that inhibits endothelial |
| | | permeability and contributes to blood vessel |
| | | maturation and stability, and may be involved |
| | | in early development of the heart |
| ANPEP | alanyl (membrane) | plays a role in the final digestion of peptides |
| | aminopeptidase | generated from hydrolysis of proteins by |
| | | gastric and pancreatic proteases |
| COL1A1 | collagen, type I, alpha 1 | a fibril-forming collagen found in most |
| | | connective tissues and is abundant in |
| | | bone, cornea, dermis and tendon |
| COL1A2 | collagen type 1, alpha 2 | fibril-forming collagen found in most |
| | | connective tissues and is abundant in bone |
| COL3A1 | collagen type 3, alpha 1 | fibrillar collagen that is found in extensible |
| | | connective tissues such as skin, lung, |
| | | uterus, intestine and the vascular system, |
| | | frequently in association with type I |
| | | collagen |
| COL4A3 | collagen, type IV, alpha | the major structural component of basement |
| | 3 (Goodpasture | membranes |
| COL5A2 | collagen type V alpha 2 | regulate the assembly of heterotypic fibers |
| | | composed of both type I and type V |
| | | collagen |
| CXCL1 | chemokine (C-X-C | plays a role in inflammation and as a |
| | motif) ligand 1 | chemoattractant for neutrophils |
| | (melanoma growth | |
| | stimulating activity, | |
| CXCL10 | chemokine (C-X-C | Binding to CXCR3 results in pleiotropic |
| | motif) ligand 10 | effects, including stimulation of monocytes, |
| | | natural killer and T-cell migration, and |
| | | modulation of adhesion molecule expression |
| CXCL8 | chemokine (C-X-C | chemoattractant and a potent angiogenic |
| | motif) ligand 8 | factor |
| DCN | Decorin | binds to type I collagen fibrils, and plays |
| | | a role in matrix assembly |
| DLX2 | distal-less homeobox 2 | postulated to play a role in forebrain and |
| | | craniofacial development |
| EDNRA | endothelin receptor type | receptor for endothelin-1, a potent |
| | A | vasoconstrictor |
| EGF | epidermal growth factor | a potent mitogenic factor that plays an |
| | | important role in the growth, proliferation |
| | | and differentiation of numerous cell types |
| EGFR | epidermal growth factor | cell proliferation and angiogenesis |
| | receptor | |
| ERBB3 | erb-b2 receptor tyrosine | cell to cell communication and cell |
| | kinase 3 | proliferation or differentiation |
| F3 | coagulation factor III | cell surface glycoprotein that enables |
| | (thromboplastin, | cells to initiate the blood coagulation |
| | tissue factor) | cascades (it functions as the high-affinity |
| | | receptor for the coagulation factor VII) |
| FGF1 | fibroblast growth factor | functions as a modifier of endothelial cell |
| | 1 | migration and proliferation, as well as |
| | | an angiogenic factor |
| GNG11 | guanine nucleotide | transmembrane signaling |
| | binding protein (G | |
| | protein), gamma 11 | |
| ICAM1 | intercellular adhesion | cell surface glycoprotein |
| | molecule 1 | |
| IFNA1 | interferon, alpha 1 | produced by macrophages and has antiviral |
| | | activity |
| IGFBP3 | insulin-like growth | it binds to IGFs in the plasma prolonging |
| | factor binding protein | the half-life of IGFs and altering their |
| | 3 | interaction with cell surface receptors |
| IL11 | interleukin 11 | stimulate the T-cell-dependent development |
| | | of immunoglobulin-producing B |
| | | cells. It also support the proliferation of |
| | | hematopoietic stem cells and |
| | | megakaryocyte progenitor cells. |
| IL1B | interleukin 1, beta | pro-inflamatory cytokine |
| IL6 | interleukin 6 | pro-inflamatory cytokine |
| ILK | integrin linked kinase | plays a role in epithelial to mesenchymal |
| | | transition and implicated in tumor |
| | | growth and metastasis |
| KRT19 | keratin 19, type I | responsible for structural integrity of |
| | | epithelial cells |
| LECT1 | leukocyte cell derived | inhibits angiogenesis and promotes |
| | chemotaxin 1 | chondrocyte growth |
| MMP1 | matrix metallopeptidase | breakdown of extracellular matrix (collagens, |
| | 1 (interstitial | types I, II, and III) |
| MMP9 | matrix metalooprotase 3 | involved in wound repair and progression of |
| | | atherosclerosis |
| NOG | Noggin | binds and inactivates members of TGF-beta |
| | | superfamily signaling proteins, such |
| | | as BMP4. It also appears to have a |
| | | pleiotropic effect |
| NPPB | natriuretic peptide B | natriuresis, diuresis, vasorelaxation, |
| | | inhibition of renin and aldosterone secretion, |
| | | and a key role in cardiovascular homeostasis |
| PDGFRA | platelet-derived growth | role in organ development, wound healing, |
| | factor receptor, | and tumor progression |
| | alpha polypeptide | |
| PLAU | plasminogen activator, | degradation of the extracellular matrix, |
| | urokinase | possible tumor cell migration and |
| | | associated with late-onset Alzheimer disease |
| PLEK2 | pleckstrin 2 | involved in cytoskeletal reorganization |
| SELE | selectin E | mediates rolling of endothelial cells |
| SERPINB2 | serpin peptidase | Also called placental PAI. Expressed in |
| | inhibitor, clade B | detectable range during pregnancy. |
| | (ovalbumin), member 2 | Cause for fibrosis during pregnancy |
| | or plasminogen | |
| | activator inhibitor type | |
| | 2 | |
| SERPINE1 | serpin peptidase | inhibitor of tissue plasminogen activator |
| | inhibitor, clade E | (tPA) and urokinase (uPA), and hence is |
| | (nexin, | an inhibitor of fibrinolysis (high concentrations |
| | plasminogen activator | of the gene product are associated with |
| | inhibitor type 1), | thrombophilia) |
| | member 1 | |
| SMAD3 | SMAD family member | transcriptional modulator activated by |
| | 3 | TGF-beta |
| SOD2 | superoxide dismutase 2, | Responsible to clear mitochondrial |
| | mitochondrial | reactive oxygen species (ROS) |
| SPP1 | secreted | causes pro-inflammatory responses |
| | phosphoprotein 1 | |
| TBX2 | T-box 2 | role in tumorigenesis as an immortalizing agent |
| TGFBI | Transforming growth | induced by TGF-beta and acts to inhibit cell |
| | factor, betainduced | adhesion |
| THBS2 | thrombospondin 2 | mediates cell-to-cell and cell-to-matrix |
| | | interactions and is a potent inhibitor of |
| | | tumor growth and angiogenesis |
| TNF | tumor necrosis factor | pro-inflamatory cytokine |
| TWIST1 | Twist homolog 1 | transcription factor involved in regulation of |
| | | endothelial to mesenchymal |
| | | transition |
| VCAN1 | Versican | important role in cell adhesion, proliferation, |
| | | migration and angiogenesis |
| WNT5A | wingless-type MMTV | regulate developmental pathway during |
| | integration site | embryogenesis |
| | family, member 5A | |
| WNT5B | wingless-type MMTV | regulate developmental pathway during |
| | integration site | embryogenesis |
| | family, member 5A | |
| ZEB2 | zinc finger E-box | transcriptional repressor that interacts with |
| | binding homeobox 2 | activated SMADs |

TABLE 5
Expression of genes (PCR array) that are strongly up regulated (>2 fold) upon
treatment with esomeprazole compared to vehicle (DMSO).

| Gene | | |
|---|---|---|
| symbol | Gene name | Function |
| ACE | angiotensin I converting | conversion of angiotensin I into |
| | enzyme | angiotensin II |
| ADGRB1 | Adhesion G Protein- | inhibitor of angiogenesis and a growth |
| | Coupled | suppressor of glioblastomas |
| | Receptor B1 | |
| AMH | anti-Mullerian hormone | causes the regression of Mullerian ducts |
| | | which would otherwise differentiate |
| | | into the uterus and fallopian tubes |
| ANG | angiogenin, | potent mediator of new blood vessel |
| | ribonuclease, RNase A | formation |
| | family, 5 | |
| APOE | apolipoprotein E | a main apoprotein of the chylomicron, |
| | | binds to a specific receptor on liver |
| | | cells and peripheral cells facilitating |
| | | chylomicron uptake |
| BMP2 | bone morphogenetic | induces bone and cartilage formation |
| | protein 2 | |
| BMP4 | bone morphogenetic | plays an important role in the onset of |
| | protein 4 | endochondral bone formation in |
| | | humans |
| CDKN1B | cyclin-dependent kinase | binds to and prevents the activation of |
| | inhibitor 1B | cyclin E-CDK2 or cyclin DCDK4 |
| | (p27, Kip1) | complexes, and thus controls the cell cycle |
| | | progression at G1. |
| | | The degradation of this protein is required |
| | | for the cellular transition from quiescence |
| | | to a proliferative state. |
| CDKN1C | cyclin-dependent kinase | Cell proliferation inhibitor and important |
| | inhibitor 1C | tumorigenic gene |
| | (p57, Kip2) | |
| CHRD | Chordin | dorsalizes early vertebrate embryonic |
| | | tissues by binding to ventralizing TGF- |
| | | beta-like bone morphogenetic proteins |
| | | and sequestering them in latent complexes |
| COL18A1 | collagen, type XVIII, | may play an important role in retinal |
| | alpha 1 | structure and in neural tube closure |
| CX3CL1 | chemokine (C-X3-C | promotes strong adhesion of leukocytes |
| | motif) ligand 1 | to activated endothelial cells |
| DSC2 | desmocollin 2 | constitute the adhesive proteins of the |
| | | desmosome cell-cell junction and are |
| | | required for cell adhesion and |
| | | desmosome formation |
| DSP | Desmoplakin | obligate component of functional |
| | | desmosomes that anchors intermediate |
| | | filaments to desmosomal plaques |
| EDN2 | endothelin 2 | secretory vasoconstrictive peptide |
| EFNA1 | ephrin-A1 | mediating developmental events, |
| | | especially in the nervous system and in |
| | | erythropoiesis |
| EFNB2 | ephrin-B2 | mediating developmental events, |
| | | especially in the nervous system and in |
| | | erythropoiesis |
| EGFR | epidermal growth factor | associated with cell proliferation |
| | receptor | |
| F11R | F11 receptor | important regulator of tight junction |
| | | assembly in epithelia |
| FGF2 | fibroblast growth factor limb | and nervous system development, wound |
| | 2 | healing, and tumor growth (possess |
| | | broad mitogenic and angiogenic activities) |
| FGFR3 | fibroblast growth factor | plays a role in bone development and |
| | receptor 3 | maintenance |
| FIGF | C-Fos Induced Growth | is active in angiogenesis, |
| | Factor | lymphangiogenesis, and endothelial |
| | (Vascular Endothelial | cell growth |
| FN1 | fibronectin 1 | involved in cell adhesion and migration |
| | | processes including embryogenesis, |
| | | wound healing, blood coagulation, |
| | | host defense, and metastasis |
| FST | Follistatin | inhibits follicle-stimulating hormone |
| | | release |
| GADD45B | growth arrest and DNA- | binding and activating MTK1/MEKK4 |
| | damageinducible, | kinase, which is an upstream |
| | beta | activator of both p38 and JNK MAPKs |
| GDF3 | growth differentiation | regulators of cell growth and |
| | factor 3 | differentiation in both embryonic and |
| | | adult tissues |
| GDF7 | growth differentiation | regulate diverse processes in growth, |
| | factor 7 | repair and embryonic development |
| ID1 | inhibitor of DNA | has no DNA binding activity but can |
| | binding 1, | inhibit the DNA binding and |
| | dominant negative | transcriptional activation ability of basic |
| | helix-loop-helix | helix-loop-helix proteins with which it |
| | protein | interacts |
| ID2 | inhibitor of DNA | inhibit the functions of basic helix-loop- |
| | binding 2, | helix transcription factors in a |
| | dominant negative | dominant-negative manner by suppressing |
| | helix-loop-helix | their heterodimerization partners |
| | protein | through the HLH domains |
| INHBB | inhibin, beta B | a pituitary FSH secretion inhibitor |
| KIT | v-kit Hardy-Zuckerman | type 3 transmembrane receptor for |
| | 4 feline | MGF (mast cell growth factor, |
| | sarcoma viral oncogene | also known as stem cell factor) |
| | homolog | |
| MDK | midkine (neurite | promotes cell growth, migration, and |
| | growth-promoting | angiogenesis, in particular during |
| | factor 2) | tumorigenesis |
| MSN | Moesin | signaling for cell-cell recognition and |
| | | cell movement |
| NODAL | nodal growth | may be essential for mesoderm formation |
| | differentiation factor subsequent | and organization of axial structures in |
| | | early embryonic development |
| NOS3 | nitric oxide synthase 3 | Nitric oxide synthesis in endothelial cells |
| | (endothelial | |
| | cell) | |
| NPR1 | natriuretic peptide | natriuresis, diuresis, vasorelaxation, |
| | receptor 1 | inhibition of renin and aldosterone |
| | | secretion, and a key role in cardiovascular |
| | | homeostasis |
| NUDT13 | nudix (nucleoside | hydrolase activity, metal ion binding |
| | diphosphate | (is localized in the mitochondrion) |
| | linked moiety X)-type | |
| | motif 13 | |
| PDGFRB | platelet-derived growth | receptor for platelet-derived growth factor, |
| | factor | and this growth factor is a mitogen for |
| | receptor, beta | cells of mesenchymal origin |
| | polypeptide | |
| STAT1 | signal transducer and | a transcription activator promoting cell |
| | activator of | viability |
| | transcription 1 | |
| TFPI | tissue factor pathway | protease inhibitor that regulates the tissue |
| | inhibitor | factor (TF)-dependent pathway of |
| | (lipoprotein-associated | blood coagulation |
| | coagulation | |
| | inhibitor) | |
| TFPI2 | tissue factor pathway | inhibit a variety of serine proteases |
| | inhibitor 2 | including factor VIIa/tissue factor, |
| | | factor Xa, plasmin, trypsin, chymotrypsin |
| | | and plasma kallikrein. It is also identified |
| | | as a tumor suppressor gene |
| TGFA | transforming growth | activates a signaling pathway for cell |
| | factor, alpha | proliferation, differentiation and |
| | | development |
| TGFB2 | transforming growth | regulate proliferation, differentiation, |
| | factor, beta 2 | adhesion and migration |
| TIE1 | tyrosine kinase with | inhibiting angiopoietin 1 signaling |
| | immunoglobulinlike | through the endothelial receptor |
| | and EGF-like domains 1 | tyrosine kinase Tie2 |
| TIMP3 | TIMP metallopeptidase | inhibitor of the matrix metalloproteinases |
| | inhibitor 3 | |
| TMEM132A | transmembrane protein | may play a role in embryonic and |
| | 132A | postnatal development of the brain. |
| | | Increased resistance to cell death induced |
| | | by serum starvation in cultured cells |
| TNFSF10 | tumor necrosis factor | pro-inflamatory cytokine |
| | (ligand) | |
| | superfamily, member 10 | |
| VCAM1 | vascular cell adhesion | mediates leukocyte-endothelial cell |
| | molecule 1 | adhesion and signal transduction |
| VWF | von Willebrand factor | antihemophilic factor carrier and a |
| | | platelet-vessel wall mediator in the |
| | | blood coagulation system |

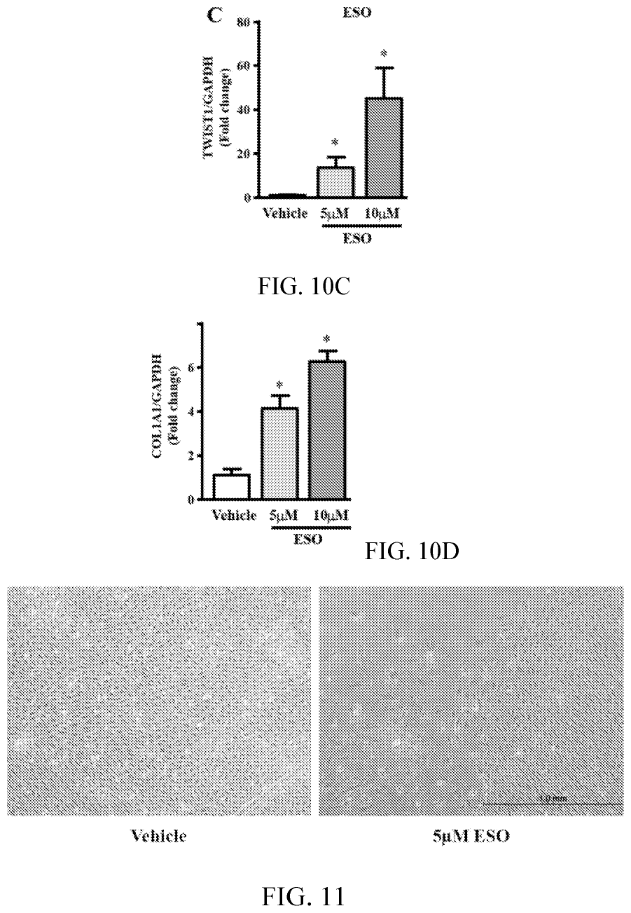

Several of these genes were selected for validation. Plasminogen activator inhibitor is a well-known marker for endothelial dysfunctions, for example, increased thrombogenicity, immune activation, oxidative stress, and senescence (Boe A E, et al., Plasminogen activator inhibitor-1 antagonist TM5441 attenuates Nω-nitro-L-arginine methyl ester-induced hypertension and vascular senescence. Circulation. 2013; 128:2318-2324. doi: 10.1161/CIRCULATIONAHA.113.003192). It was found that plasminogen activator inhibitor message and protein expression were upregulated in esomeprazole-treated cells (FIG. 3G-3I). It was also found that genes associated with EndoMT, including TWIST1, COL1A1, and SMAD3 (FIG. 10A-10C), were upregulated, together with a decline in the expression of von Willebrand factor (FIG. 10D), a marker for vascular endothelium. In additional studies, after treating ECs with esomeprazole (5 or 10 μmol/L) or vehicle for 3 passages, treatment was discontinued and the ECs were maintained in an endothelial growth medium at the same passage for ≈3 months. At the 3-month time point, the ECs that had been exposed to vehicle remained confluent, with occasional apoptotic and senescent cells. By contrast, there was a qualitative difference in the cells that had been exposed to esomeprazole, with most high-power fields, showing some cell loss or EndoMT (FIG. 11). To conclude, chronic exposure to a PPI induces endothelial dysfunction consistent with EndoMT and senescence.

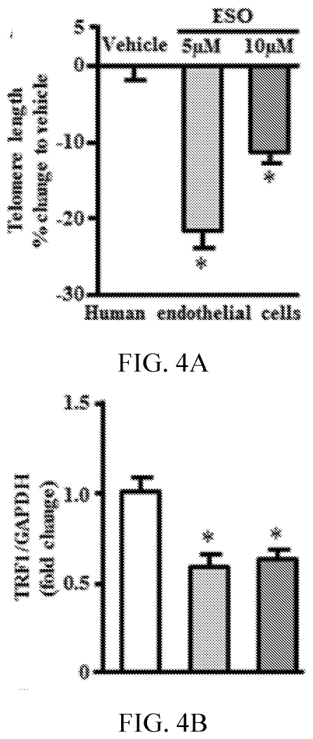

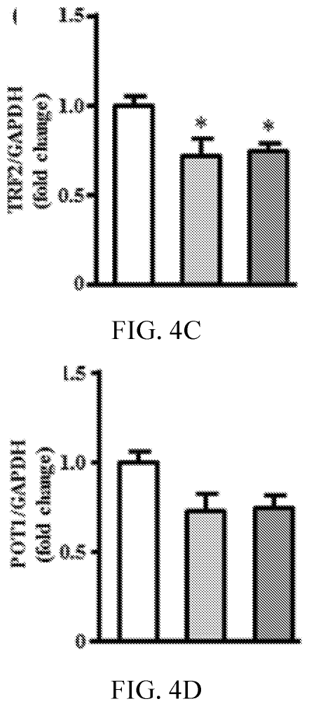

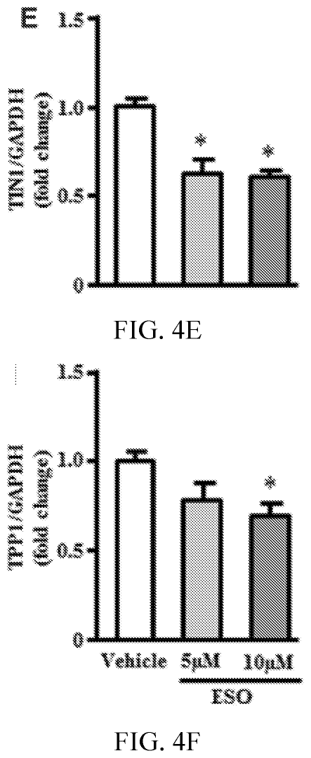

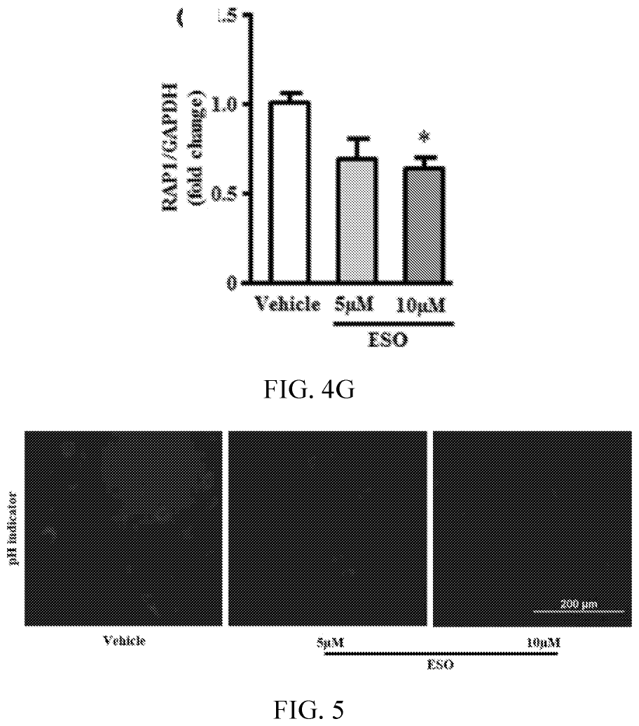

Endothelial senescence is associated with attrition of telomere length (Fyhrquist F, et al., The roles of senescence and telomere shortening in cardiovascular disease. Nat Rev Cardiol. 2013; 10:274-283. doi: 10.1038/nrcardio.2013.30), whereas restoration of EC telomere length can reverse senescence-associated endothelial dysfunction (Matsushita H, et al., eNOS activity is reduced in senescent human endothelial cells: Preservation by hTERT immortalization. Circ Res. 2001; 89:793-798. doi: 10.1161/hh2101.098443; Ramunas J, et al., Transient delivery of modified mRNA encoding TERT rapidly extends telomeres in human cells. FASEB J. 2015; 29:1930-1939. doi: 10.1096/fj.14-259531). As expected, neither group of ECs manifested telomerase expression or activity (FIG. 12). Using monochrome multiplex quantitative polymerase chain reaction as previously described (Ramunas J, et al., Transient delivery of modified mRNA encoding TERT rapidly extends telomeres in human cells. FASEB J. 2015; 29:1930-1939. doi: 10.1096/fj.14-259531), a significant decrease in telomere length was observed in esomeprazole-treated group compared with vehicle (FIG. 4A). To assess the mechanism of telomere shortening, the expression of genes involved in regulating the shelterin complex were examined. The shelterin complex is encoded by 6 genes (TRF1, TRF2, POT1, RAP1, TIN2, and TPP1) involved in regulation and maintenance of telomere length and function (de Lange T. Shelterin: the protein complex that shapes and safeguards human telomeres. Genes Dev. 2005; 19:2100-2110. doi: 10.1101/gad.1346005). A global downregulation of all 6 genes of the shelterin complex was observed (FIG. 4C-4H), which could explain, in part, the effect of the PPI to accelerate telomere erosion.

### Discussion

The salient findings of this study are that long-term exposure to proton pump inhibition (1) impairs lysosomal acidification and enzyme activity, in association with protein aggregate accumulation; (2) increases the generation of reactive oxygen species and impairs the NO synthase pathway; (3) accelerates telomere erosion in association with reduced expression of the shelterin complex; and (4) speeds endothelial aging as manifested by impaired cell proliferation and angiogenesis, together with histological markers of senescence and EndoMT. Lysosomes bind to autophagosomes to complete the process of autophagy (Gatica D, et al., Molecular mechanisms of autophagy in the cardiovascular system. Circ Res. 2015; 116:456-467. doi: 10.1161/CIRCRESAHA.114.303788), which comprises the degradation and elimination of unwanted cellular products, including misfolded proteins (Ohkuma S, Poole B. Fluorescence probe measurement of the intralysosomal pH in living cells and the perturbation of pH by various agents. Proc Natl Acad Sci USA. 1978; 75:3327-3331; Liu W, et al., Inhibition of lysosomal enzyme activities by proton pump inhibitors. J Gastroenterol. 2013; 48:1343-1352. doi:10.1007/s00535-013-0774-5). An impairment of lysosomal acidification and reduced lysosomal enzyme activity might be expected to result in an accumulation of protein aggregates.

The studies were conducted in a clinically relevant dose range. In adults, the peak plasma concentration (Cmax) of esomeprazole is 4.7 μmol/L with the 40-mg dose (NEXIUM (esomeprazole magnesium) label. http://www.accessdata.fda gov/drugsatfda_docs/label/2014/022101s014021957s017021153s0501b1.pdf. Reference ID: 3675799. Accessed Dec. 29, 2014; Shin J M, Kim N. Pharmacokinetics and pharmacodynamics of the proton pump inhibitors. J Neurogastroenterol Motil. 2013; 19:25-35. doi:10.5056/jnm.2013.19.1.25). The metabolism of esomeprazole is dependent on the isoenzyme CYP2C19, which exhibits polymorphism. About 3% of whites and 23% of Asians are poor metabolizers and may experience a 3-fold increase in plasma concentration of esomeprazole (Klotz U, Schwab M, Treiber G. CYP2C19 polymorphism and proton pump inhibitors. Basic Clin Pharmacol Toxicol. 2004; 95:2-8. doi:10.1111/j.1600-0773.2004.pto950102.x; NEXIUM (esomeprazole magnesium) label. http://www.accessdata.fda gov/drugsatfda_docs/label/2014/022101s014021957s017021153s0501b1.pdf. Reference ID: 3675799. Accessed Dec. 29, 2014).

In addition, chronic PPI exposure upregulated genes that are involved in EndoMT and was associated with histological changes consistent with EndoMT. EndoMT is a feature of senescent ECs and may itself play an important role in cardiovascular disease, as well as other disorders characterized by fibrosis and loss of the microvasculature. Furthermore, it was shown that esomeprazole downregulates the expression of the shelterin complex genes, in association with a reduction in telomere length. An observation of clinical importance is that ranitidine, an alternative treatment for gastroesophageal reflux disease, which acts by a different mechanism than the PPIs, does not have an adverse effect on endothelial aging.

Chronic exposure of human ECs to the PPIs, esomeprazole or SCH-28080, accelerated endothelial aging. This adverse effect seems to be because of an inhibition of lysosomal acidification and subsequent impairment of proteostasis. The accumulation of protein aggregates is associated with an increase in oxidative stress, endothelial dysfunction, and senescence. Vascular senescence would provide a mechanistic explanation for the accumulating evidence that PPIs increase the risk of cardiovascular morbidity and mortality, renal failure, and dementia. In the presence of consistent epidemiological evidence of harm and a unifying mechanism for the disparate disorders linked to PPI use and with the knowledge that PPIs are being used by millions of people for indications and durations that were never tested or approved, it is time for the pharmaceutical industry and regulatory agencies to revisit the specificity and the safety of these agents.

1. A method of screening for one or more agents that inhibit senescence, the method comprising:
(a) culturing mammalian cells with one or more proton pump inhibitors, wherein the proton pump inhibitors promote senescence of the mammalian cells;
(b) contacting the culture of step (a) with one or more candidate agents;
(c) assaying the mammalian cells for one or more positive indicators and/or for one or more negative indicators of senescence, a decrease in the level of one or more positive indicators or an increase in the level of one or more negative indicators of senescence in the presence of the one or more candidate agent, as compared to a control culture lacking the one or more candidate agents, indicates the candidate agent inhibits senescence.

2. The method of claim 1, wherein the one or more proton pump inhibitors is present in the culture at a concentration of 1 to 20 μmol/L.

3. The method of claim 1, wherein the one or more proton pump inhibitors are selected from the group consisting of esomeprazole, lansoprazole, dexlansoprazole, omeprazole, pantoprazole, rabeprazole, and ilaprazole.

4. The method of claim 3, wherein the proton pump inhibitor is esomeprazole.

5. The method of claim 1, wherein the culture is assayed by microscopy, fluorescence assay, colorimetric assay, sequencing, microarray, immunoassay, Western blot, Northern blot, qPCR, RT-PCR, or any combination thereof.

6. The method of claim 1, wherein the positive indicator of senescence is selected from the group consisting of an increase in lysosomal pH, an increase in protein aggregation, an increase in superoxide anion, an increase in expression of cell cycle inhibitors, an increase in expression of plasminogen activator inhibitor, an increase in senescence-associated beta-galactosidase positive cells, an increase in elongated spindle-shaped cells, and any combination thereof.

7. The method of claim 1, wherein the negative indicator of senescence is selected from the group consisting of a decrease in lysosomal enzyme activity, a decrease in nitric oxide levels, a decrease in nitrate levels, a decrease in activity of the NO synthase pathway, a decrease in replicative capacity of the cells, a decrease in angiogenic capacity, a change in morphology, a decrease in telomere length, reduced expression of the shelterin complex, a decrease in the mitotic index, and any combination thereof.

8. The method of claim 1, wherein the candidate agent is a peptide, nucleic acid, small molecule or any combination thereof.

9. The method of claim 1, wherein the mamalian cells are selected from the group consisting of endothelial cells, keratinocytes, and fibroblast cells.

10. A method of screening for one or more agents that promote senescence, the method comprising:
(a) providing a first culture of mammalian cells and one or more proton pump inhibitors, wherein the proton pump inhibitors promote senescence of the mammalian cells;
(b) assaying the first culture for one or more positive and/or negative indicators of senescence;
(c) providing a second culture of mammalian cells and one or more candidate agents;
(d) assaying the second culture of mammalian cells for the same positive and/or negative indicators of senescence, detection of one or more of the same positive and/or negative indicators of senescence in the second culture as compared to the first culture indicating the one or more candidate agents promotes senescence.

11. The method of claim 10, wherein the one or more proton pump inhibitors in the first and second cultures is present in a concentration of 1 to 20 μmol/L.

12. The method of claim 10, wherein the one or more proton pump inhibitors are selected from the group consisting of lansoprazole, dexlansoprazole, omeprazole, esomeprazole, pantoprazole, rabeprazole, and ilaprazole.

13. The method of claim 12, wherein the proton pump inhibitor is esomeprazole.

14. The method of claim 10, wherein the first and second cultures are assayed by microscopy, a fluorescence assay, a colorimetric assay, sequencing, microarray, an immunoassay, Western blot, Northern blot, qPCR, RT-PCR, or any combination thereof.

15. The method of claim 10, wherein the positive indicator of senescence is selected from the group consisting of an increase in lysosomal pH, an increase in protein aggregation, an increase in superoxide anion, an increase in expression of cell cycle inhibitors, an increase in expression of plasminogen activator inhibitor, an increase in senescence-associated beta-galactosidase positive cells, and any combination thereof.

16. The method of claim 10, wherein the negative indicator of senescence is selected from the group consisting of a decrease in lysosomal enzyme activity, a decrease in nitric oxide levels, a decrease in nitrate levels, a decrease in activity of the NO synthase pathway, a decrease in cell proliferation, a decrease in angiogenic capacity, a change in morphology, a decrease in telomere length, reduced expression of the shelterin complex, a decrease in the mitotic index, and any combination thereof.

17. The method of claim 10, wherein the one or more candidate agents are selected from the group consisting of a peptide, nucleic acid, small molecule, and any combination thereof.

18. The method of claim 10, wherein the mammalian cells are selected from the group consisting of endothelial cells, fibroblast cells and keratinocytes.

19. A method of promoting senescence of a proliferative cell comprising contacting the proliferative cell with a composition comprising an effective amount of one or more proton pump inhibitors, wherein the proliferative cell is not a tumor cell.

20. The method of claim 19, wherein the composition is formulated for topical administration.

21. The method of claim 19, wherein the composition is formulation for ocular, oral, inhalation, intravenous, intrathecal, intra-uterine, intraperitoneal, intravesical, intra-articular, intramuscular or subcutaneous administration.

22. The method of claim 19, wherein the proliferative cell is a skin cell or a vascular cell.

23. The method of claim 20, wherein the composition comprises 1 to 20 μm of the one or more proton pump inhibitors.

24. The method of claim 22, wherein the proliferative cell is a enodthelial cell, keratinocyte, or fibroblast cell.

25. A kit comprising an mammalian cell line and one or more proton pump inhibitors.

26. The kit of claim 25, wherein the one or more proton pump inhibitors are selected from the group consisting of lansoprazole, dexlansoprazole, omeprazole, esomeprazole, pantoprazole, rabeprazole, and ilaprazole.

27. The kit of claim 26, wherein the proton pump inhibitor is esomeprazole.

28. The kit of claim 25, wherein the kit further comprises one or more reagents for assaying an indicator of senescence.

29. The kit of claim 25, wherein the kit further comprises reagents for inducing senescence.

30. The kit of claim 25, wherein the mammalian cell line is selected from the group consisting of an endothealial cell line, a fibroblast cell line or a keratinocyte cell line.

31. The kit of claim 25, wherein the mammalian cell line is selected from the group consisting of human umbilical venous endothelial cells, human aortic endothelial cells, human coronary artery endothelial cells, human microvascular endothelial cells.
